(19) United States
(12) Patent Application Publication
(10) Pub. No.: US 2022/0050110 A1
(43) Pub. Date: Feb. 17, 2022

(54) USE OF MCM5 AS A MARKER IN SEMEN FOR PROSTATE CANCER

(71) Applicant: ARQUER DIAGNOSTICS LIMITED, Wearfield, Sunderland (GB)

(72) Inventors: Jacqueline STOCKLEY, Wearfield, Sunderland (GB); Cheryl NYBERG, Wearfield, Sunderland (GB)

(21) Appl. No.: 17/413,878

(22) PCT Filed: Dec. 19, 2019
(86) PCT No.: PCT/GB2019/053627

§ 371 Date: Jun. 14, 2021

(30) Foreign Application Priority Data
Dec. 20, 2018 (GB) 1820868.6

(51) Int. Cl. G01N 33/574

(52) U.S. Cl. CPC G01N 33/57434

(57) ABSTRACT

The present invention relates to a method for detecting the presence or absence of a prostate cancer in a subject, the method comprising steps of: providing a semen sample obtained from the subject; and detecting at least one biomarker or determining the concentration of at least one biomarker. The present invention also relates to lysis buffers, monoclonal antibodies, and kits that can be used in such methods.

### FIELD OF THE INVENTION

The present invention relates to methods for detecting the presence or absence of a prostate cancer in a subject, and the use of a lysis buffer in said method. The present invention also relates to the use of a first monoclonal antibody and/or a second monoclonal antibody in a method of detecting the presence or absence of prostate cancer in a subject. The present invention further relates to a kit comprising a lysis buffer, a first monoclonal antibody and/or a second monoclonal antibody, and the use of said kit in the method of detecting the presence or absence of prostate cancer in a subject.

### BACKGROUND OF THE INVENTION

There is an unmet medical need for methods for diagnosing prostate cancers at an early stage. Prostate cancer is the most common cancer in men with over 47,000 new cases diagnosed each year in the UK; about 11,500 deaths annually are caused by prostate cancer. There are around 450,000 new cases in Europe, 210,000 in the US and 1,250,000 worldwide annually. Cancer Research UK report that a quarter of all new cases of cancer diagnosed in men in the UK are prostate cancers and 60% of new diagnoses are in men aged over 70 years. The most common form of the disease is adenocarcinoma. The 5-year survival rate is almost 80% in the UK. Those with close relatives with prostate or breast cancer are more at risk of developing the disease. Black males have an increased risk of prostate cancer. There is some evidence for a possible relationship between agricultural pesticide exposure and incidence of prostate cancer.

The symptoms of prostate cancer are similar to those caused by benign enlargement of the prostate gland, and include urgency to urinate, difficulty or pain in passing urine and rarely, blood in the urine or semen. However, in many men the disease remains symptomless until painful metastases form, predominantly in the bones.

Treatment depends on the stage and grade of the tumour and the patient's general health and age. Options include active surveillance, partial or radical prostatectomy, orchidectomy, hormone treatment, and radiotherapy such as brachytherapy. Orchidectomy and hormone treatment reduce or eliminate the production of testosterone, which is essential for tumour growth.

The definite diagnosis of prostate cancer requires a multi-faceted approach. The current gold standard diagnostic test for prostate cancer is the histological examination of biopsy material. The decision to biopsy is based on age-related serum PSA level and/or an abnormal digital rectal examination (DRE). DRE, in which the gland is palpated trans-rectally to examine for abnormal morphology is also non-specific. Tumours that are too small to alter the morphology of the gland will not be detected, and abnormal morphology or enlargement is also caused by non-malignant conditions. This is a problem in the art. Samples of the prostate gland are commonly taken using TRUS (trans-rectal ultra sound)-guided needle biopsy. A number of needle cores are taken, typically up to 12, in order to maximize the area of the gland sampled. The procedure is carried out in the outpatients department under local anaesthesia by a urologist with the aid of a nurse or healthcare assistant. This procedure suffers from drawbacks including being somewhat painful for the patient, and exposing the patient to a risk of sepsis and/or bleeding. The tissue cores are microscopically examined in a laboratory for the presence of malignant cells, which has the problem of being labour intensive and requiring highly trained cytologists, as well as being vulnerable to human error.

It can be appreciated that biopsies are invasive and costly. There is a need in the art for a more cost-effective, reliable and/or non-invasive tool for the diagnosis and/or surveillance of urological cancer such as prostate cancer. Known alternate and/or less invasive diagnostic procedures for prostate cancer involve the analysis of specific biological markers (‘biomarkers’). There follows a real unmet need for a screening tool for prostate cancer which can detect prostate cancer with high specificity and sensitivity.

### SUMMARY OF THE INVENTION

The present invention provides methods, uses and kits for the early detection of prostate cancer without the need for invasive surgical procedures. The methods of the invention can be performed using semen samples, and are therefore suitable for use in the clinical laboratory and/or for point-of-care applications. Furthermore, the methods of the present invention are easy to carry out as they do not require complicated processing steps.

In particular, the present inventors have demonstrated that prostate cancer can be readily detected directly in semen samples with high accuracy. These results demonstrate the detection of prostate cancer in semen samples has clear potential both as a diagnostic test in a symptomatic population and as a screening tool to identify prostate cancers within a symptomatic population. Ultimately, this enables earlier diagnosis and treatment, which is known to improve survival in prostate cancer.

Thus, in a first aspect, the present invention provides a method for detecting the presence or absence of a prostate cancer in a subject, the method comprising steps of providing a semen sample obtained from the subject and detecting at least one biomarker or determining the concentration of at least one biomarker.

In a second aspect, the present invention provides a use of a lysis buffer of the invention in a method of detecting the presence or absence of prostate cancer in a subject.

In a third aspect, the present invention provides a use of a first monoclonal antibody of the invention and/or a second monoclonal antibody of the invention in a method of detecting the presence or absence of prostate cancer in a subject.

In a fourth aspect, the present invention provides a use of a kit in a method of detecting the presence or absence of prostate cancer in a subject, wherein the kit comprises:

a) a lysis buffer of the invention;

b) a first monoclonal antibody of the invention; and/or

c) a second monoclonal antibody of the invention.

In a fifth aspect, the present invention provides a kit comprising:

a) a lysis buffer of the invention;

b) a first monoclonal antibody of the invention; and/or

c) a second monoclonal antibody of the invention; and

d) instructions for use of the lysis buffer and/or the first monoclonal antibody and/or the second monoclonal antibody in a method of detecting the presence or absence of prostate cancer in a sample.

### BRIEF DESCRIPTION OF THE DRAWINGS

FIG. 1 is a Box Plot of MCM5 concentrations as determined from semen samples in normal versus prostate cancer patients.

FIG. 2 shows a Box Plot of MCM5 concentrations as determined from semen samples in normal versus prostate cancer patients broken down by grade group.

FIG. 3 is a sequence listing.

### DETAILED DESCRIPTION

### Definitions

The term “comprises” (comprise, comprising) should be understood to have its normal meaning in the art, i.e. that the stated feature or group of features is included, but that the term does not exclude any other stated feature or group of features from also being present. For example, a lysis buffer comprising a detergent may contain other components.

The term “consists of” should also be understood to have its normal meaning in the art, i.e. that the stated feature or group of features is included, to the exclusion of further features. For example a lysis buffer consisting of a detergent contains detergent and no other components. A lysis buffer comprising a detergent consisting of polysorbate 80 may comprise components other than detergents but the only detergent in the lysis buffer is polysorbate 80.

For every embodiment in which “comprises” or “comprising” is used, we anticipate a further embodiment in which “consists of” or “consisting of” is used. Thus, every disclosure of “comprises” should be considered to be a disclosure of “consists of”. Sequence Homology/Identity

Although sequence homology can also be considered in terms of functional similarity (i.e., amino acid residues having similar chemical properties/functions), in the context of the present document it is preferred to express homology in terms of sequence identity.

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids). For comparison over longer sequences, gap scoring is used to produce an optimal alignment to accurately reflect identity levels in related sequences having insertion(s) or deletion(s) relative to one another. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Typically sequence comparisons are carried out over the length of the reference sequence. For example, if the user wished to determine whether a given sequence is 95% identical to SEQ ID NO: 27, SEQ ID NO: 27 would be the reference sequence. For example, to assess whether a sequence is at least 80% identical to SEQ ID NO: 27 (an example of a reference sequence), the skilled person would carry out an alignment over the length of SEQ ID NO: 27, and identify how many positions in the test sequence were identical to those of SEQ ID NO: 27. If at least 80% of the positions are identical, the test sequence is at least 80% identical to SEQ ID NO: 27. If the sequence is shorter than SEQ ID NO: 27, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

For the purposes of the present invention, the term “fragment” refers to a contiguous portion of a reference sequence. For example, a fragment of SEQ ID NO: 27 of 50 amino acids in length refers to 50 contiguous amino acids of SEQ ID NO: 27.

In the context of the present application, a homologous amino acid sequence is taken to include an amino acid sequence which is at least 40, 50, 60, 70, 80 or 90% identical to the reference sequence. Most suitably a polypeptide having at least 90% sequence identity to the biomarker of interest will be taken as indicative of the presence of that biomarker; more suitably a polypeptide which is 95% or more suitably 98% identical at the amino acid level will be taken to indicate presence of that biomarker. Suitably said comparison is made over at least the length of the polypeptide or fragment which is being assayed to determine the presence or absence of the biomarker of interest. Most suitably the comparison is made across the full length of the polypeptide of interest.

A method for detecting the presence or absence of a prostate cancer in a subject

The present invention provides a method for detecting the presence or absence of a prostate cancer in a subject.

Typically, it is not possible for a diagnostic method to predict with 100% accuracy whether or not prostate cancer is present in a subject. Thus, the wording “detecting the presence or absence of a prostate cancer in a subject” can be understood to refer to determining whether prostate cancer is likely to be present or absent in a subject. If prostate cancer is likely to be present, the subject may wish to investigate medical intervention.

Prostate Cancer

For the purposes of the present invention, the term “prostate cancer” refers to a condition in which cancer cells form in the tissues of the prostate gland. In some embodiments, the prostate cancer is an adenocarcinoma, a carcinoma in situ, or a prostatic intraepithelial neoplasia.

The prostate cancer may be characterised by anaplasia (e.g. poor cellular differentiation and/or the loss of morphological characteristics associated with healthy cells), invasiveness (i.e. the ability to spread into a healthy tissue), metastasis (i.e. the ability to spread from an initial site to different secondary site), and/or genome instability.

In some embodiments, the prostate cancer is malignant. The cells of malignant prostate cancer may be characterised by anaplasia (e.g. poor cellular differentiation and/or the loss of morphological characteristics associated with healthy cells), invasiveness (i.e. the ability to spread into a healthy tissue), metastasis (i.e. the ability to spread from an initial site to different secondary site), and/or genome instability.

In some embodiments, the prostate cancer is low grade, such as grade 1-3. In some embodiments, the prostate cancer is higher grade, such as grade 4/5. The methods of the present invention may be used to detect a low grade prostate cancer, such as a prostate cancer of grade 4 or 5. The methods of the present invention may be used to detect a high grade prostate cancer, such as a prostate cancer of grade 1-3. High grade prostate cancers may be associated with excessive cell proliferation rates, typically longer cell lifespans and poor cellular differentiation. The earlier prostate cancer is detected the better, and the present method is advantageous as it may be used to detect low grade cancers. Grading systems used in cancer biology and medicine categorize cancer cells to with respect to their lack of cellular differentiation. This reflects the extent to which the cancer cells differ in morphology from healthy cells found in the tissue from which the cancer cell originated. The grading system can be used to provide an indication of how quickly a particular cancer might be expected to grow.

Typically, the Gleason grading system is used to grade prostate cancer. This system is widely used in cancer medicine. Briefly however, to determine the Gleason grade of a prostate cancer sample, a Gleason score is first assigned to the sample by visually inspecting cell morphology using a microscope and grading the two most common cell types as Grades 1-5 based on how abnormal the cells appear. Grade 1 means that the cells look almost like normal prostate cells. Grade 5 means that the cells look very different from normal prostate cells. The Gleason score is then determined by adding together the two most common grades. For example, if the most common grade of the cells in the sample was Grade 3, and the second most common was Grade 4, the Gleason score for this sample would be 7. Higher Gleason scores are indicative of a faster growing prostate cancer that is more likely to spread. The Gleason score can be used to assign the prostate cancer to a Gleason grade group as set out below:
Grade group 1: Gleason score 6 or lower (low-grade cancer)
Grade group 2: Gleason score 3+4=7 (medium-grade cancer)
Grade group 3: Gleason score 4+3=7 (medium-grade cancer)
Grade group 4: Gleason score 8 (high-grade cancer)
Grade group 5: Gleason score 9 to 10 (high-grade cancer).

Subjects

The subject is preferably an animal subject and even more preferably a human. However, in some embodiments, the subject may be a non-human mammal such as a primate, feline, canine, bovine, equine, murine, ovine, or porcine.

The subject is preferably male. In some embodiments, the subject is middle-aged, for example between 40 and 65, or between 45 and 60 years old. In some embodiments, the subject is elderly, for example at least 65, 70, 75, 80, 85, or 90 years in age.

In some embodiments, the subject is suffering from prostate cancer. In some embodiments, the subject is in remission from prostate cancer. In some embodiments, the subject is suspected of having prostate cancer. In some embodiments, the subject appears healthy. In some embodiments, the subject is at risk of developing prostate cancer. In some embodiments, the subject has an increased risk of developing prostate cancer.

The subject may be at risk or have an increased risk of developing prostate cancer where the subject has been previously identified as carrying a genetic marker indicating an increased risk of developing prostate cancer. The subject may be at risk or have an increased risk of developing prostate cancer where the subject has a family history of gynaecological cancer. The subject may be at risk or have an increased risk of developing prostate cancer where the subject is suffering from, or has previously been diagnosed with a cancer, for example, a non-prostate cancer.

In some embodiments, the subject is suffering from one or more symptoms associated with prostate cancer. Symptoms associated with prostate cancer include difficult starting and maintaining steady urine streams, urinary urgency, painful urination (dysuria), increased urination at night (nocturia), increased urination frequency, painful bowel movements, blood in urine (haematuria), blood in the semen (haematospermia), constipation, back pain, pelvic pain, and/or a sore hip.

Providing a Semen Sample Obtained from the Subject

The methods of the invention comprise providing a semen sample obtained from the subject. The semen sample may be obtained via ejaculation or surgical sperm retrieval. Preferably, the semen sample is obtained via ejaculation. In some embodiments, the semen sample is obtained after a period of abstinence. For the purposes of the present application, a “period of abstinence” is a period in which the subject has not ejaculated (e.g. a period in which the subject has not masturbated or engaged in sexual intercourse). Optionally, the period of abstinence is at least 12 hours, at least 1 day, at least 3 days, less than 1 week, between 12 hours and 1 week, between 1 day and 1 week, or between 3 days and 1 week.

Biomarkers

The methods of the invention comprise a step of detecting at least one biomarker or determining the concentration of at least one biomarker. Biological markers, or biomarkers, are molecules found in blood or other body fluids or tissues that help to indicate a biological state, process, event, condition or disease. It will be clear to those skilled in the art that biomarkers could consist of, but are not limited to DNA, RNA, chromosomal anomalies, proteins and their derivatives, or metabolites. In general terms, a biomarker may be regarded as a distinctive biological or biologically-derived indicator of a process, event or condition. In some embodiments, biomarkers may be specific proteins or peptides whose occurrence, over-expression or under-expression in biological fluids such as semen may reflect the existence, progression, response to treatment or severity of cancer, such as prostate cancer.

Thus the term “biomarker” includes all biologically relevant forms of the protein or nucleic acid identified, including post-translationally modified polypeptides. For example, when the biomarker is a polypeptide the marker protein can be present in the sample in a glycosylated, phosphorylated, multimeric or precursor form.

Optionally, the biomarker comprises or consists of an MCM protein. MCM proteins 2-7 comprise part of the pre-replication complexes which form on chromatin and which are essential prerequisites, or licensing factors, for subsequent DNA replication. The MCM protein complexes act as replicative helicases and thus are core components of the DNA replication machinery. MCM proteins are upregulated in the transition from the GO to Gl/S phase of the cell cycle and actively participate in cell cycle regulation. The MCM proteins form an annular structure around the chromatin.

The human MCM5 gene maps to 22q13.1 and the mature MCM5 protein consists of 734 amino acids (SEQ ID NO: 35; UNIPROT P33992: HUMAN DNA replication licensing factor MCM5). The term “MCM5” refers to a polypeptide of SEQ ID NO: 35, or a polypeptide 85%, 90%, 95%, 98%, or 100% identity to SEQ ID NO: 35.

Detecting at Least One Biomarker or Determining the Concentration of at Least One Biomarker

The skilled person may use any suitable technique known in the art to detect the at least one biomarker or determine the concentration of the at least one biomarker. For example, the at least one biomarker may be detected or the concentration of the at least one biomarker may be determined by interaction with a ligand or ligands, 1-D or 2-D gel-based analysis systems, liquid chromatography, combined liquid chromatography and any mass spectrometry techniques including MSMS, ICAT® or iTRAQ®, agglutination tests, thin-layer chromatography, NMR spectroscopy, sandwich immunoassays, enzyme linked immunosorbent assays (ELISAs), radioimmunoassays (RAI), enzyme immunoassays (EIA), lateral flow/immunochromatographic strip tests, Western Blotting, immunoprecipitation, particle-based immunoassays including using gold, silver, or latex particles and magnetic particles or Q-dots, or any other suitable technique known in the art.

In some embodiments of the methods of the invention, determining the concentration of the at least one biomarker or determining whether the concentration of the biomarker is higher than a pre-defined cut-off is carried out using an ELISA assay. In a further embodiment, the ELISA assay is a sandwich ELISA assay. A sandwich ELISA assay comprises steps of capturing the at least one biomarker to be detected or whose concentration is to be determined using a “capture antibody” already bound to a plate, and detecting how much of the at least one biomarker has been captured using a “detection antibody”. The detection antibody may be pre-conjugated to a label such as the enzyme HRP (Horse Radish Peroxidase). The ELISA plate may then be exposed to the labelled detection antibody, such that the labelled detection antibody binds to the captured at least one biomarker. After exposure to the labelled detection antibody, the ELISA plate should then be washed to remove any excess unbound labelled detection antibody. The washed plate can then be exposed to an agent whose properties are changed by the label (of the detection antibody) in a measurable manner. The concentration of the detection antibody may then be determined or the biomarker may be detected. For example, if the detection antibody is labelled by e.g. conjugation to HRP, the ELISA plate may be exposed to 3,3′,5,5′-Tetramethylbenzidine (TMB) substrate. The concentration of the detection antibody, and therefore the concentration of the at least one biomarker in the original non-invasive sample, may then be determined by quantitation of the colour change corresponding to the conversion of TMB into a coloured product. The “capture antibody” may be the first monoclonal antibody or the second monoclonal antibody described herein. Similarly, the “detection antibody” may be the first monoclonal antibody or the second monoclonal antibody described herein.

Detecting the at least one biomarker may be qualitative step. For example, a step of detecting the at least one biomarker may be performed without determining a numerical value for the concentration of the at least one biomarker in the prostate cancer, such as by merely determining whether the concentration of the biomarker is above or below a pre-defined cut-off or reference. Optionally, detecting the at least one biomarker comprises determining whether the concentration of the biomarker is higher than a pre-defined cut-off. For example, in a qualitative ELISA assay, the intensity of a coloured product (e.g. a coloured product produced by an enzyme linked to a detection antibody) may be compared to a threshold colour intensity. In this embodiment, the intensity of the coloured product is indicative of the concentration of the at least one biomarker without the need to determine a numerical value for the concentration of the at least one biomarker. The reference used in a qualitative ELISA of this type may be a previously determined threshold intensity of the coloured product corresponding to a pre-defined cut-off.

In some embodiments, the at least one biomarker is an MCM protein, and the step of determining the concentration of the MCM protein comprises performing an ELISA assay. In some embodiments, the at least one biomarker is an MCM protein, and the step of determining the concentration of the MCM protein comprises performing a sandwich ELISA assay. In some embodiments, the at least one biomarker is MCM5 protein, and the step of determining the concentration of the MCM5 protein comprises performing an ELISA assay. In some embodiments, the biomarker is MCM5 protein, and the step of determining the concentration of the MCM5 protein comprises performing a sandwich ELISA assay.

Determining Whether the Concentration of the Biomarker is Higher than a Pre-Defined Cut-Off

Optionally, detecting the at least one biomarker comprises determining whether the concentration of the biomarker is higher than a pre-defined cut-off. Prostate cancer may be likely to be present if the concentration of the biomarker is higher than the pre-defined cut-off.

The concentration of the at least one biomarker may be said to be higher than the cut-off if there is a statistically significant difference between the concentration of the at least one biomarker and the pre-defined cut-off For example, prostate cancer is likely to be present when the concentration of the at least one biomarker is higher than the pre-defined cut-off by at least one, at least two, at least three, or at least four standard deviations.

Optionally, as mentioned above, detecting the at least one biomarker may be a qualitative step, i.e. may be performed without determining a numerical value for the concentration of the at least one biomarker in the prostate cancer.

The pre-defined cut-off may be between 40 pg/mL and 100 pg/mL, between 50 pg/mL and 75 pg/mL, between 60 pg/mL and 70 pg/mL, or around 65 pg/mL.

Comparing the Concentration of the at Least One Biomarker to a Reference

In some embodiments, the concentration of the at least one biomarker is compared to a reference and prostate cancer is likely to be present if the concentration of the at least one biomarker is abnormal compared to or higher than the reference.

The concentration of the at least one biomarker may be said to be abnormal compared to the reference if there is a statistically significant difference between the concentration of the at least one biomarker and the reference. For example, the concentration of the at least one biomarker is abnormal if it is significantly lower than the reference. Alternatively, the concentration of the least one biomarker is abnormal if it is significantly higher than the reference. In one embodiment of the invention, prostate cancer is likely to be present if the concentration of the at least one biomarker is higher than the reference. For example, where the reference is an average concentration of the at least one biomarker, prostate cancer is likely to be present when the concentration of the at least one biomarker is higher than the reference by at least one, at least two, at least three, or at least four standard deviations.

Suitably, the reference may be a concentration of the at least one biomarker determined for a semen sample obtained from one or more healthy individuals. A healthy individual is one who is not presently suffering from a prostate cancer. For example, the healthy individual may be one who has never suffered from a prostate cancer or who currently has not symptoms of a prostate cancer.

Where the reference is the concentration of the at least one biomarker determined for a sample prepared from one or more healthy individuals, the reference may be an average concentration of the at least one biomarker determined for multiple samples obtained from a single healthy individual. Alternatively, the reference may be an average concentration of the at least one biomarker determined for multiple samples prepared from multiple healthy individuals. In this context, “one or more samples” may be at least 10, 100, 500, 1000, 10 000, 100 000 or 1 000 000 samples.

In one embodiment, the reference can be an average concentration of the at least one biomarker determined from one or more samples prepared from one or more healthy individuals in parallel with detecting the at least one biomarker or determining the concentration of the at least one biomarker released from the semen sample obtained in the methods of the invention, e.g. the semen sample being subject to a method of the present invention for detecting the presence or absence of prostate cancer.

In another embodiment, the reference can be an average concentration of the at least one biomarker previously determined for one or more samples prepared from a healthy individual. In such embodiments, a numeric comparison may be made by comparing the concentration of the at least one biomarker determined for the semen sample obtained in the invention to the reference. This avoids having to duplicate the analysis by determining a reference in parallel each time a semen sample from a subject is analysed.

Suitably, the reference may be matched to the subject being analysed e.g. by age, by ethnic background or other such criteria which are well known in the art. For example, the reference may suitably be matched to specific patient sub-groups e.g. elderly subjects, or those with a previous relevant history such as a predisposition to prostate cancer.

Suitably, the sample used to derive the reference was obtained in the same manner as the sample obtained in the method of the invention. For example, if the sample obtained in the method of the invention is a semen sample obtained after 5 days of abstinence, then the reference may be a semen sample obtained after 5 days of abstinence from an apparently healthy individual.

In some embodiments, the concentration of the at least one biomarker determined may be compared to a concentration of the at least one biomarker previously determined from a semen sample obtained from the same subject. In these embodiments, the previously determined concentration of the at least one biomarker is used as the reference. This can be beneficial in monitoring the possibility of recurrence in the subject, which can in turn be beneficial in monitoring the course and/or effectiveness of a treatment of the subject.

Diagnosing Prostate Cancer

The method may be a method for diagnosing prostate cancer, and the method may further comprise a step of diagnosing the subject as having prostate cancer if the concentration of the biomarker is higher than the pre-defined cut-off. The method may be a method for diagnosing prostate cancer, and the method may further comprise a step of diagnosing the subject as having prostate cancer if the concentration of the at least one biomarker is higher than the reference.

If the subject is diagnosed as having prostate cancer, the method may comprise a further step of treating the subject for prostate cancer. For example, the method may comprise treating the subject using radical prostatectomy (surgery to remove the prostate), external beam radiotherapy, permanent seed brachytherapy, hormone therapy, high dose-rate brachytherapy, high intensity focused ultrasound, cryotherapy, chemotherapy, or administration of a prostate cancer drug such as abiraterone, enzalutamide, cabazitaxel, docetaxel, bisphosphonates, or radium-223.

Sensitivity and Specificity

The methods of the present invention allow prostate cancer to be detected with high specificity and high sensitivity. In the field of medical diagnostics and as used herein the term “sensitivity” (also referred to as the true positive rate) refers to a measure of the proportion of actual positives that are correctly identified as such. In other words, the sensitivity of a diagnostic test may be expressed as the number of true positives i.e. individuals correctly identified as having a disease as a proportion of all the individuals having the disease in the test population (i.e. the sum of true positive and false negative outcomes). Thus, a high sensitivity diagnostic test is desirable as it rarely misidentifies individuals having the disease. This means that a negative result obtained by a highly sensitive test has a high likelihood of ruling out the disease.

In the field of medical diagnostics, and as used herein, the term “specificity” (also referred to as the true negative rate) refers to a measure of the proportion of actual negatives that are correctly identified as such. In other words, the specificity of a diagnostic test may be expressed as the number of true negatives (i.e. healthy individuals correctly identified as not having a disease) as a proportion of all the healthy individuals in the test population (i.e. the sum of true negative and false positive outcomes). Thus, a high specificity diagnostic test is desirable as it rarely misidentifies healthy individuals. This means that a positive result obtained by a highly specific test has a high likelihood of ruling in the disease.

In the field of medical diagnostics, and as used herein, the term “positive predictive value (PPV)” refers to the proportion of all positive outcomes generated by a diagnostic test that are true positive outcomes. Put another way, PPV can be defined as the number of true positive outcomes divided by the sum of true positive outcomes and false positive outcomes. In the field of medical diagnostics, and as used herein, the term “negative predictive value (NPV)” refers to the proportion of all negatives outcomes generated by a diagnostic test that are true negative outcomes. Put another way, NPV can be defined as the number of true negative outcomes divided by the sum of true negative outcomes and false negative outcomes.

In the field of medical diagnostics, and as used herein, a “Receiver Operating Characteristic (ROC) curve” refers to a plot of true positive rate (sensitivity) against the false positive rate (1−specificity) for all possible cut-off values. In the field of medical diagnostics, and as used herein, a “Youdens index” refers to the cut-off point at which the distance between the ROC curve and the line of chance (45 degree line) is highest. These terms are well known in the art and to the skilled person.

The specificity and/or sensitivity of a method may be determined by performing said method on samples which are known to be positive samples (e.g. samples from patients having prostate cancer) and/or samples which are known to be negative samples (e.g. samples from healthy individuals). The extent to which the method correctly identifies the known positive samples (i.e. the sensitivity/true positive rate of the method) and/or the known negative samples (i.e. the specificity/true negative rate of the method) can thus be determined.

Optionally, the method of the invention has a sensitivity of greater than 40%, greater than 50%, greater than 60%, between 40% and 100%, between 50% and 100%, between 60% and 100%, or around 61%. Optionally, the method of the invention has a specificity of greater than 70%, greater than 75%, between 70% and 100%, between 75% and 100%, or around 79%. Optionally, the method has a positive predictive value (PPV) of greater than 60%, greater than 70%, greater than 75%, between 60% and 100%, between 70% and 100%, between 75% and 100%, or around 78%. Optionally, the method has a negative predictive value (NPV) of greater than 55%, greater than 60%, between 55% and 100%, between 60% and 100%, or around 63%. Optionally, the method of the invention has a sensitivity of greater than 60%, a specificity of greater than 75%, a PPV of greater than 75%, and an NPV of greater than 60%. Optionally, the method of the invention has a sensitivity of between 50% and 100%, a specificity of between 75% and 100%, a PPV of between 75% and 100%, and an NPV of between 60% and 100%.

In embodiments where detecting the at least one biomarker comprises determining whether the concentration of the biomarker is abnormal compared to a pre-defined cut-off, the value of the pre-defined cut-off will affect the sensitivity and specificity of the method. If a higher pre-defined cut-off is used, the specificity will increase, but the sensitivity will decrease. Preferably, the pre-defined cut-off that is selected achieves a balance of good sensitivity and specificity, but the user may wish to vary the pre-defined cut-off depending on whether high sensitivity or high specificity is important for a particular application.

Optionally, the method of the invention has a sensitivity of greater than 40%, greater than 50%, greater than 60%, between 40% and 100%, between 50% and 100%, between 60% and 100%, or around 61% using a pre-defined cut-off of 65 pg/ml. Optionally, the method has a specificity of greater than 70%, greater than 75%, between 70% and 100%, between 75% and 100%, or around 79% using a pre-defined cut-off of 65 pg/mL. Optionally, the method has a positive predictive value (PPV) of greater than 60%, greater than 70%, greater than 75%, between 60% and 100%, between 70% and 100%, between 75% and 100% or around 78% using a pre-defined cut-off of 65 pg/mL. Optionally, the method has a negative predictive value (NPV) of greater than 55%, greater than 60%, between 55% and 100%, between 60% and 100%, or around 63% using a pre-defined cut-off of 65 pg/mL. Optionally, the method of the invention has a sensitivity of between 50% and 100%, a specificity of between 75% and 100%, a PPV of between 75% and 100%, and an NPV of between 60% and 100% using a pre-defined cut-off of 65 pg/mL.

The present application demonstrates that the presence of absence of prostate cancer may be determined more accurately (e.g. with greater sensitivity or specificity) compared to an assay using a urine sample. Thus, optionally, the method may have a greater sensitivity, specificity, NPV and/or PPV than an equivalent method carried out using a urine sample. Optionally, the method has a greater sensitivity and specificity than an equivalent method carried out using a urine sample. Optionally, the method has a greater sensitivity and NPV than an equivalent method carried out using a urine sample. Optionally, the method has a greater sensitivity and PPV than an equivalent method carried out using a urine sample. Optionally, the method has a greater specificity and NPV than an equivalent method carried out using a urine sample. Optionally, the method has a greater specificity and PPV than an equivalent method carried out using a urine sample. Optionally, the method has a greater NPV and PPV than an equivalent method carried out using a urine sample. Optionally, the method has a greater sensitivity, specificity, NPV and PPV than an equivalent method carried out using a urine sample. Optionally, the equivalent method using a urine sample uses a cut-off of 3 pg/mL.

Treating the Semen Sample to Release the at Least One Biomarker from Cells in the Semen Sample

Normal epithelial cells in the prostate gland do not express MCM5 as they are fully differentiated and therefore epithelial cells shed into prostatic secretions do not contain MCM5. However, in the presence of a tumour, non-differentiated immature proliferating cells which express MCM5 are present at the surface epithelium lining the prostate gland, and these MCM5-expressing cells can be shed into prostatic secretions. Accordingly, upon ejaculation, the presence of MCM5 in the semen is indicative of a prostate cancer. Thus, to detect the presence of absence of prostate cancer in a semen sample, it is advantageous to be able to release the MCM5 from the epithelial cells in the semen to allow detection using, for example, an antibody based ELISA. For these reasons, the method may comprise a step of treating the semen sample to release the at least one biomarker from cells in the semen sample. The step of treating the semen sample to release the at least one biomarker may comprise exposing the semen sample to a lysis buffer capable of releasing the at least one biomarker from cells in the semen sample. Optionally, exposing the semen sample to a lysis buffer capable of releasing the at least one biomarker from cells in the semen sample is carried out on a pellet of the cells in the semen sample obtained by centrifuging the semen sample. Optionally, exposing the semen sample to a lysis buffer capable of releasing the at least one biomarker from cells in the semen sample is carried out on a supernatant sample obtained by centrifuging the semen sample. Preferably, the step of treating the semen sample to release the at least one biomarker from cells in the semen sample is carried out before the step of detecting the at least one biomarker or determining the concentration of the at least one biomarker. Optionally, exposing the semen sample to a lysis buffer capable of releasing the at least one biomarker from cells in the semen sample comprises:

passing the non-invasive sample through a filter for capturing cells, such that cells are captured in the filter;

passing a lysis buffer through the filter, such that the captured cells are exposed to the lysis buffer; and/or

incubating the filter for a period of time, such that the lysis buffer causes the cells to release at least one biomarker.

Lysis buffers are generally buffers which are able to lyse cells. In addition to releasing one or more biomarkers from cells, the lysis buffer may be compatible with the method used for subsequent analysis. For example, where the analysis method is double-antibody sandwich ELISA, it may be desirable that the lysis buffer does not degrade the capture antibody bound to the surface of the microtitre plate. Lysis buffers generally but not exclusively comprise one or more detergents (also known as surfactants), one or more salts and a buffering agent. In some instance, the concentrations of these components may affect the efficacy of the lysis buffer.

In one embodiment, a buffer can be said to be a “lysis buffer” if it is capable of lysing at least 10%, 20%, 30%, 40%, 50%, 60%, 70%, 80%, 90%, 91%, 92%, 93%, 94%, 95%, 96%, 97%, 98%, 99%, or substantially all of the cells in a semen sample. In one embodiment, a buffer can be said to be a “lysis buffer” if it is capable of lysing at least 10%, 20%, 30%, 40%, 50%, 60%, 70%, 80%, 90%, 91%, 92%, 93%, 94%, 95%, 96%, 97%, 98%, 99%, or substantially all of the cells in a semen sample where the cells in the non-invasive sample are exposed to the lysis buffer for at least 1 minute, 5 minutes, 10 minutes, 20 minutes, 30 minutes, 60 minutes, 1 hour, 2 hours, 6 hours, 12 hours, 24 hours, or overnight. In one embodiment, a buffer can be said to be a “lysis buffer” if it is capable of lysing at least 10%, 20%, 30%, 40%, 50%, 60%, 70%, 80%, 90%, 91%, 92%, 93%, 94%, 95%, 96%, 97%, 98%, 99%, or substantially all of the cells in a semen sample where the cells in the non-invasive sample are exposed to the lysis buffer for at least 1 minute, 5 minutes, 10 minutes, 20 minutes, 30 minutes, 60 minutes, 1 hour, 2 hours, 6 hours, 12 hours, 24 hours, or overnight at 4° C., 20° C., or room temperature.

In one embodiment, the lysis buffer is capable of releasing the at least one biomarker, such as an MCM protein or MCM5, from cells in the semen sample. A lysis buffer will be considered to be “capable of releasing a biomarker from cells in the semen sample” if the amount of the biomarker (such as MCM5) released is greater than 40%, 50%, 60%, 70%, or 80% the amount released when a buffer comprising 25 mM Tris pH 7.6, 150 mM sodium chloride, 1% sodium deoxycholate, 0.1% sodium dodecyl sulphate (SDS), and 1% Triton-X100 is used. The antibodies used in the assay should be antibodies that bind to the biomarker. For example, where the biomarker is MCM5, a first monoclonal antibody and/or second monoclonal antibody that bind to MCM5 could be used. Preferably 12A7 and 4B4 antibodies are used.

Optionally, the lysis buffer is capable of releasing MCM5 from cells in the semen sample and does not denature the MCM5 protein. A lysis buffer does not denature the MCM5 protein if at least 40%, 50%, 60%, 70%, or 80% of the MCM5 is intact. Whether or not MCM5 protein is intact or denatured may be determined by exposing MCM5 to antibodies 12A7 and 4B4 as defined herein. Without being bound by theory, it is thought that antibodies 12A7 and 4B4 bind to MCM5 in its intact (i.e. non-denatured state), and so an ELISA assay using antibodies 12A7 and 4B4 can be used to determine the proportion of MCM5 which is denatured.

Optionally, the lysis buffer does not denature an antibody. This can be useful where detecting the at least one biomarker or determining the concentration of at least one biomarker comprises detecting using an antibody such as by an ELISA assay. If the lysis buffer does not denature an antibody, then it does not need to be removed before the step of detecting the at least one biomarker or determining the concentration of at least one biomarker. A lysis buffer can be considered to not denature an antibody if the activity of the antibody after exposure to the lysis buffer is at least 40%, 50%, 60%, 70%, or 80% the activity of the antibody prior to exposure to the lysis buffer. The activity of the antibody may be tested using an ELISA assay such as that described in Example 1.

In some embodiments, the lysis buffer comprises a detergent (also referred to as a surfactant). In general detergents are compounds that are known to disrupt cell walls. Detergents are amphiphilic having both hydrophobic and hydrophilic regions. Suitable detergents are well known to the person of skill in the art. Examples of detergents that may suitably be used in a lysis buffer according to the present invention include, but are not limited to, Triton X-100, sodium doceyl sulphate (SDS), and sodium deoxycolate,

Optionally, the detergent comprises Triton X-100. Optionally, the detergent comprises or consists of sodium deoxycholate. Optionally, the detergent comprises or consists of sodium dodecyl sulphate (SDS). Optionally, the detergent comprises Triton X-100, sodium doceyl sulphate (SDS), and sodium deoxycolate. Optionally, the detergent comprises Triton X-100 at a concentration between 0.01% and 25%, between 0.01% and 10%, between 0.05% and 5%, between 0.1% and 2%, between 0.5% and 2%, between 0.75% and 1.25%, or about 1%. Optionally, the detergent comprises or consists of sodium deoxycholate at a concentration between 0.1% and 20%, between 0.1 and 10%, between 0.1 and 5%, between 0.5% and 5%, between 0.5% and 2.5%, between 0.75% and 2.5%, between 0.75% and 1.25%, or about 1%. Optionally, the detergent comprises or consists of sodium dodecyl sulphate (SDS) at a concentration between 0.001% and 10%, between 0.01% and 5%, between 0.05% and 5%, between 0.01% and 1%, between 0.05% and 1%, between 0.05% and 0.5%, between 0.075% and 0.25%, or about 0.1%. Optionally, the detergent consists of between 0.5% and 2% of Triton X-100, between 0.5% and 2% of sodium deoxycholate, and between 0.05% and 0.5% of sodium dodecyl sulphate (SDS).

In some embodiments, the lysis buffer comprises a buffer component. A buffer component can be considered to be any component which maintains the pH of the lysis buffer at a pH varying by less than 2.0 pH units, 1.5 pH units, or 1.0 pH units. The buffer component may have a pH of between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6; and/or maintains the pH of the lysis buffer at between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6. For example, the buffer component preferably has a pH between pH 7.4 and pH 7.8.

An example of a buffer that may suitably be used in a lysis buffer according to the present invention is Tris. Optionally, the buffer component comprises or consists of Tris. Optionally, the buffer component comprises or consists of Tris at a concentration greater than 1 mM, between 1 mM and 350 mM, between 5 and 200 mM, between 5 and 100 mM, between 5 and 50 mM, between 5 mM and 40 mM, between 5 mM and 35 mM, between 10 mM and 35 mM, between 15 mM and 35 mM, between 15 mM and 30 mM, between 20 mM and 30 mM, or about 25 mM. The buffer component may consist of Tris at a concentration of between 15 mM and 35 mM.

In some embodiments, the lysis buffer comprises a salt. Various salts are well known to the person skilled in the art. In some embodiments, the salt is added to the lysis buffer to provide a particular level of ionic strength. An example of a salt that can be suitably used in a lysis buffer according to the invention is sodium chloride. Optionally, the lysis buffer comprises sodium chloride at a concentration between 10 mM and 350 mM, between 20 mM and 300 mM, between 50 mM and 250 mM, between 100 mM and 250, between 100 mM and 200 mM, between 125 mM and 175 mM, or about 150 mM. Optionally, the lysis buffer comprises sodium chloride at a concentration of between 100 mM and 200 mM.

Optionally, the lysis buffer comprises:

(i) between 1 mM and 100 mM Tris;

(ii) between 50 mM and 300 mM sodium chloride;

(iii) between 0.1 and 5% sodium deoxycholate;

(iv) between 0.01 and 1% sodium dodecyl sulphate; and/or

(v) between 0.1 and 5% Triton-X100.

Optionally, the lysis buffer comprises:

(i) between 10 mM and 40 mM Tris;

(ii) between 100 mM and 200 mM sodium chloride;

(iii) between 0.5 and 2% sodium deoxycholate;

(iv) between 0.05 and 0.5% sodium dodecyl sulphate; and/or

(v) between 0.5 and 2% Triton-X100.

Optionally, the lysis buffer comprises:

(i) about 25 mM Tris;

(ii) about 150 mM sodium chloride;

(iii) about 1% sodium deoxycholate;

(iv) about 0.1% sodium dodecyl sulphate; and/or

(v) about 1% Triton-X100.

Antibodies

The step of detecting the at least one biomarker or determining the concentration of the at least one biomarker may comprise:

exposing the semen sample to a first monoclonal antibody and/or a second monoclonal antibody; and

detecting the at least one biomarker bound to the first monoclonal antibody and/or the second monoclonal antibody or determining the concentration of the at least one biomarker bound to the first monoclonal antibody and/or the second monoclonal antibody.

The term “antibody” can refer to naturally occurring forms or recombinant antibodies such as single-chain antibodies, chimeric antibodies or humanised antibodies. The terms “antibody” and “antibodies” may also be considered to encompass fragments of antibodies that can bind to a target protein, such as an MCM protein like MCM5. Such fragments may include Fab′₂, F′(ab)₂, Fv, single chain antibodies or diabodies. The antibodies may be naturally occurring, full length antibodies (rather than fragments). In a further embodiment, the antibodies are not humanised antibodies.

In general, antibodies are formed from two heavy chains and two lights chains. Each heavy chain is made up of heavy chain constant region (CH) and a heavy chain variable region (VH). Similarly each light chain is made up of light chain constant region (CL) and a light chain variable region (VL). The VH and VL regions comprise complementarity defining regions (CDRs). The CDRs are, primarily responsible for specific binding to the target protein.

It is well within the ability of the person skilled in the art to develop an antibody that binds to a biomarker protein of interest, such as MCM5. This may be performed by immunising a mammal such as a mouse, rabbit or guinea pig, with the biomarker protein e.g. MCM5.

It may be beneficial to include an adjuvant such as Freund's complete adjuvant. The spleen cells of the immunised mammal are removed and fused with myeloma cells to form hybridoma lines which are immortal given appropriate conditions and which secrete antibodies. The hybridomas are separated into single clones and the antibodies secreted by each clone are evaluated for their binding ability to the biomarker protein (e.g. MCM5).

First Monoclonal Antibody and Second Monoclonal Antibody

In some embodiments of the methods of the invention, the semen sample is exposed to a first monoclonal antibody and/or a second monoclonal antibody. As discussed above, such embodiments may involve performing an ELISA assay or a sandwich ELISA assay which comprises exposing the semen sample to first monoclonal antibody described herein and/or second monoclonal antibody described herein. In one embodiment, the first monoclonal antibody and/or the second monoclonal antibody bind to MCM5. Preferably, the first monoclonal antibody and/or the second monoclonal antibody bind specifically to MCM5. Preferably, the first monoclonal antibody binds to SEQ ID NO: 1. Preferably the second monoclonal antibody binds to SEQ ID NO: 2.

In some embodiments, the first monoclonal antibody or the second monoclonal antibody will bind to an epitope (fragment) of MCM5 (for example SEQ ID NO: 1 or SEQ ID NO: 2). Thus, the term “antibody which binds to MCM5” refers to an antibody that binds to only a single epitope of MCM5, such as SEQ ID NO: 1 or SEQ ID NO: 2.

For the purposes of the present invention the term ‘binding affinity’ refers to the ability of an antibody to bind to its target. For the purposes of the present invention, the term ‘specifically binds’ refers to an antibody that binds to a target, such as MCM5, with a binding affinity that is at least 2-fold, 10-fold, 50-fold or 100-fold greater than its binding affinity for binding to another non-target molecule. In an embodiment the non-target molecule is an MCM protein, other than MCM5, such as MCM2. Preferably, the first monoclonal antibody and/or the second monoclonal antibody is capable of binding to an MCM protein, optionally MCM5, with a binding affinity that is at least 2-fold, 10-fold, 50-fold, or 100-fold greater than its binding affinity for binding to another non-target molecule. Even more preferably, the first monoclonal antibody and/or the second monoclonal antibody is capable of binding to SEQ ID NO: 1 (such as the first monoclonal antibody) or SEQ ID NO: 2 (such as the second monoclonal antibody) with a binding affinity that is at least 2-fold, 10-fold, 50-fold, or 100-fold greater than its binding affinity for binding to another non-target molecule.

A preferred method for the evaluation of binding affinity for MCM5 is by ELISA. Preferably, the first monoclonal antibody and/or the second monoclonal antibody have an affinity for MCM5 (measured as an EC50 or 50% maximum binding concentration, as described in Example 2) of 2500 ng/ml or lower, 1500 ng/ml or lower, 1000 ng/ml or lower, 600 ng/ml or lower, 50 ng/ml or lower, 30 ng/ml or lower, 20 ng/ml or lower, or 10 ng/ml or lower. The EC50 will typically be higher than 1 ng/ml and thus the EC50 may be between 1 ng/ml and any of the upper limits specified in the preceding sentence. Other standard assays to evaluate the binding ability of ligands such as antibodies towards targets are known in the art, including for example, Western blots, RIAs, and flow cytometry analysis. The binding kinetics (e.g. binding affinity) of the antibody also can be assessed by standard assays known in the art, such as by Surface Plasmon Resonance (SPR) (e.g. Biacore™ system) analysis. The affinity constant (KD) for binding to MCM5 is preferably in the range of 0.01-10 nM, 0.01-5 nM, 0.01-1 nM, 0.01-0.5 nM, 0.01-0.25 nM, 0.025-0.25 nM, or 0.04-0.25 nM. In one embodiment, the first and/or second monoclonal antibody used in the ELISA has an affinity for MCM5 of about 0.01 nM, 0.02 nM, 0.03 nM, 0.04 nM, 0.05 nM, 0.06 nM, 0.07 nM, 0.08 nM, 0.09 nM, 0.1 nM, 0.2 nM, 0.3 nM, 0.4 nM, or 0.5 nM. In one embodiment, the first monoclonal antibody and/or second monoclonal antibody used in the ELISA has an affinity for MCM5 of about 0.05 nM. In one embodiment, the first monoclonal antibody and/or second monoclonal antibody used in the ELISA has an affinity for MCM5 of about 0.2 nM. In one embodiment, the first monoclonal antibody and/or second monoclonal antibody used in the ELISA has an affinity for MCM5 of about 0.233 nM. In one embodiment, the first monoclonal antibody has an affinity for MCM5 of about 0.05 nM and the second monoclonal antibody has an affinity for MCM5 of about 0.2 nM. In one embodiment, the first monoclonal antibody has an affinity for MCM5 of about 0.05 nM and the second monoclonal antibody has an affinity for MCM5 of about 0.233 nM. In one embodiment, the first monoclonal antibody is 12A7 and has an affinity for MCM5 of about 0.05 nM and the second monoclonal antibody is 4B4 and has an affinity for MCM5 of about 0.2 nM. In one embodiment, the first monoclonal antibody is 12A7 and has an affinity for MCM5 of about 0.05 nM and the second monoclonal antibody is 4B4 and has an affinity for MCM5 of about 0.233 nM. The association rate (ka) is preferably in the range of 0.4-3.4×10⁶ 1/M. The dissociation rate (kd) is preferably in the range of 1-10×10⁻³ 1/s. These values may typically be determined by SPR (surface plasmon resonance).

Antibody Complementary Determining Regions (CDRs)

The methods of the present invention may comprise the use of a first monoclonal antibody and/or a second monoclonal antibody comprising at least one of the CDRs of antibodies 12A7 or 4B4, i.e. a CDR selected from the group consisting of:
a. 12A7 CDRH1 which has a sequence of SEQ ID NO: 9 or a sequence that differs from SEQ ID NO: 9 by 1, 2 or 3 amino acid substitutions;
b. 12A7 CDRH2 which has a sequence of SEQ ID NO: 11 or a sequence that differs from SEQ ID NO: 11 by 1, 2 or 3 amino acid substitutions;
c. 12A7 CDRH3 which has a sequence of SEQ ID NO: 13 or a sequence that differs from SEQ ID NO: 13 by 1, 2 or 3 amino acid substitutions;
d. 12A7 CDRL1 which has a sequence of SEQ ID NO: 3 or a sequence that differs from SEQ ID NO: 3 by 1, 2 or 3 amino acid substitutions;
e. 12A7 CDRL2 which has a sequence of SEQ ID NO: 5 or a sequence that differs from SEQ ID NO: 5 by 1, 2 or 3 amino acid substitutions;
f. 12A7 CDRL3 which has a sequence of SEQ ID NO: 7 or a sequence that differs from SEQ ID NO: 7 by 1, 2 or 3 amino acid substitutions;
g. 4B4 CDRH1 which has a sequence of SEQ ID NO: 21 or a sequence that differs from SEQ ID NO: 21 by 1, 2 or 3 amino acid substitutions;
h. 4B4 CDRH2 which has a sequence of SEQ ID NO: 23 or a sequence that differs from SEQ ID NO: 23 by 1, 2 or 3 amino acid substitutions;
i. 4B4 CDRH3 which has a sequence of SEQ ID NO: 25 or a sequence that differs from SEQ ID NO: 25 by 1, 2 or 3 amino acid substitutions
j. 4B4 CDRL1 which has a sequence of SEQ ID NO: 15 or a sequence that differs from SEQ ID NO: 15 by 1, 2 or 3 amino acid substitutions;
k. 4B4 CDRL2 which has a sequence of SEQ ID NO: 17 or a sequence that differs from SEQ ID NO: 17 by 1, 2 or 3 amino acid substitutions; and
l. 4B4 CDRL3 which has a sequence of SEQ ID NO: 19 or a sequence that differs from SEQ ID NO: 19 by 1, 2 or 3 amino acid substitutions.

Antibodies that have the same CDRs as the 4B4 and 12A7 antibodies may differ substantially from the sequences of 4B4 and 12A7 in other regions. Such antibodies may, for example, be antibody fragments.

The phrase “sequence that differs from SEQ ID NO: 3 by a single amino acid substitution” refers to the possibility of replacing one amino acid defined in SEQ ID NO: 3 by a different amino acid. Preferably such a replacement is a conservative amino acid substitution. The following eight groups each contain amino acids that are typically conservative substitutions for one another (1) Alanine, Glycine; (2) Aspartic acid, Glutamic acid; (3) Asparagine, Glutamine; (4) Arginine, Lysine; (5) Isoleucine, Leucine, Methionine, Valine; (6) Phenylalanine, Tyrosine, Tryptophan; (7) Serine, Threonine; and (8) Cysteine, Methionine.

In an embodiment, the first monoclonal antibody comprises at least one CDR from the heavy chain of 12A7 (12A7 CDRH1, 12A7 CDRH2 or 12A7 CDRH3) as well as at least one CDR from the light chain of 12A7 (12A7 CDRL1, 12A7 CDRL2 or 12A7 CDRL3). In a further embodiment, the first monoclonal antibody comprises at least two CDRs from the heavy chain of 12A7 and at least two CDRs from the light chain of 12A7. In a preferred embodiment, the first monoclonal antibody comprises all three CDRs from the heavy chain of 12A7 and/or all three CDRs from the light chain of 12A7. In an embodiment, the first monoclonal antibody comprises 12A7 CDRL1 and 12A7 CDRL2, 12A7 CDRL1 and 12A7 CDRL3, 12A7 CDRL1 and 12A7 CDRH1, 12A7 CDRL1 and 12A7 CDRH2, 12A7 CDRL1 and 12A7 CDRH3, 12A7 CDRL2 and 12A7 CDRL3, 12A7 CDRL2 and 12A7 CDRH1, 12A7 CDRL2 and 12A7 CDRH2, 12A7 CDRL2 and 12A7 CDRH3, 12A7 CDRL3 and 12A7 CDRH1, 12A7 CDRL3 and 12A7 CDRH2, 12A7 CDRL3 and 12A7 CDRH3, 12A7 CDRH1 and 12A7 CDRH2, 12A7 CDRH1 and 12A7 CDRH3, or 12A7 CDRH2 and 12A7 CDRH3.

In an embodiment, the second monoclonal antibody comprises at least one CDR from the heavy chain of 4B4 (4B4 CDRH1, 4B4 CDRH2 or 4B4 CDRH3) as well as at least one CDR from the light chain of 4B4 (4B4 CDRL1, 4B4 CDRL2 or 4B4 CDRL3). In a further embodiment, the second monoclonal antibody comprises at least two CDRs from the heavy chain of 4B4 and at least two CDRs from the light chain of 4B4. In a preferred embodiment, the second monoclonal antibody comprises all three CDRs from the heavy chain of 4B4 and/or all three CDRs from the light chain of 4B4. In an embodiment, the second monoclonal antibody comprises 4B4 CDRL1 and 4B4 CDRL2, 4B4 CDRL1 and 4B4 CDRL3, 4B4 CDRL1 and 4B4 CDRH1, 4B4 CDRL1 and 4B4 CDRH2, 4B4 CDRL1 and 4B4 CDRH3, 4B4 CDRL2 and 4B4 CDRL3, 4B4 CDRL2 and 4B4 CDRH1, 4B4 CDRL2 and 4B4 CDRH2, 4B4 CDRL2 and 4B4 CDRH3, 4B4 CDRL3 and 4B4 CDRH1, 4B4 CDRL3 and 4B4 CDRH2, 4B4 CDRL3 and 4B4 CDRH3, 4B4 CDRH1 and 4B4 CDRH2, 4B4 CDRH1 and 4B4 CDRH3, or 4B4 CDRH2 and 4B4 CDRH3. In a preferred embodiment, an antibody comprises at least one CDR having a sequence identical to that described in any one of SEQ ID NO: 3 (12A7 CDRL1), SEQ ID NO: 5 (12A7 CDRL2), SEQ ID NO: 7 (12A7 CDRL3), SEQ ID NO: 9 (12A7 CDRH1), SEQ ID NO: 11 (12A7 CDRH2), SEQ ID NO: 13 (12A7 CDR H3), SEQ ID NO: 15 (4B4 CDRL1), SEQ ID NO: 17 (4B4 CDRL2), SEQ ID NO: 19 (4B4 CDRL3), SEQ ID NO: 21 (4B4 CDRH1), SEQ ID NO: 23 (4B4 CDRH2) or SEQ ID NO: 25 (4B4 CDRH3). In an embodiment, where the first monoclonal antibody comprises 12A7 CDRL2, the 12A7 CDRL2 has the sequence described in SEQ ID NO: 5. In a further embodiment, where the first monoclonal antibody comprises 12A7 CDRL1, the 12A7 CDRL1 has the sequence described in SEQ ID NO: 3. In a further embodiment, where the first monoclonal antibody comprises 12A7 CDRL3, the 12A7 CDRL3 has the sequence described in SEQ ID NO: 7. In a further embodiment, where the first monoclonal antibody comprises 12A7 CDRH1, the 12A7 CDRH1 has the sequence described in SEQ ID NO: 9. In a further embodiment, where the first monoclonal antibody comprises 12A7 CDRH2, the 12A7 CDRH2 has the sequence described in SEQ ID NO: 11. In a further embodiment, where the first monoclonal antibody comprises 12A7 CDRH3, the 12A7 CDRH3 has the sequence described in SEQ ID NO: 13. In a further embodiment, where the second monoclonal antibody comprises 4B4 CDRL1, the 4B4 CDRL1 has the sequence described in SEQ ID NO: 15. In a further embodiment, where the second monoclonal antibody comprises 4B4 CDRL2, the 4B4 CDRL2 has the sequence described in SEQ ID NO: 17. In a further embodiment, where the second monoclonal antibody comprises 4B4 CDRL3, the 4B4 CDRL3 has the sequence described in SEQ ID NO: 19. In a further embodiment, where the second monoclonal antibody comprises 4B4 CDRH1, the 4B4 CDRH1 has the sequence described in SEQ ID NO: 21. In a further embodiment, where the second monoclonal antibody comprises 4B4 CDRH2, the 4B4 CDRH2 has the sequence described in SEQ ID NO: 23. In a further embodiment, where the second monoclonal antibody comprises 4B4 CDRH3, the 4B4 CDRH3 has the sequence described in SEQ ID NO: 25.

Preferably, the first monoclonal antibody and/or the second monoclonal antibody comprises at least one of the CDRs of 12A7 or 4B4 binds (optionally specifically binds) to MCM5. Even more preferably, the first monoclonal antibody and/or the second monoclonal antibody comprising at least one of the CDRs of 12A7 or 4B4 binds (optionally specifically binds) to SEQ ID NO: 1 or SEQ ID NO: 2.

Heavy and Light Chain Variable Regions

In some embodiments, the first monoclonal antibody used in the present invention comprises a heavy chain variable region having a sequence at least 85%, 90%, 95%, 98%, 99% or 100% identical to SEQ ID NO: 29. In some embodiments, the first monoclonal antibody used in the present invention comprises a light chain variable region sequence having a sequence at least 85%, 90%, 95%, 98%, 99% or 100% identical to SEQ ID NO: 27. In some embodiments, the second monoclonal antibody used in the present invention comprises a heavy chain variable region having a sequence at least 85%, 90%, 95%, 98%, 99% or 100% identical to SEQ ID NO: 33. In some embodiments, the second monoclonal antibody used in the present invention comprises a light chain variable region sequence having a sequence at least 85%, 90%, 95%, 98%, 99% or 100% identical to SEQ ID NO: 31. Such antibodies may be referred to as “variant antibodies”.

In an embodiment, the first monoclonal antibody comprises a heavy chain variable region having a sequence at least 95% identical to SEQ ID NO: 29. In a further embodiment, the first monoclonal antibody comprises a heavy chain variable region having a sequence at least 98% identical to SEQ ID NO: 29. In one embodiment, the first monoclonal antibody comprises a light chain variable region having a sequence at least 95% identical to SEQ ID NO: 27. In a further embodiment, the first monoclonal antibody comprises a light chain variable region having a sequence at least 98% identical to SEQ ID NO: 27. In a further embodiment, the first monoclonal antibody comprises a light chain variable region having a sequence at least 95% identical to SEQ ID NO: 27 and a heavy chain variable region having a sequence at least 95% identical to SEQ ID NO: 29. In a preferred embodiment, the first monoclonal antibody comprises a heavy chain variable region having a sequence at least 98% identical to SEQ ID NO: 29 and a light chain variable region having a sequence at least 98% identical to SEQ ID NO: 27.

In a further embodiment, the second monoclonal antibody comprises a heavy chain variable region having a sequence at least 95% identical to SEQ ID NO: 33. In a further embodiment, the second monoclonal antibody comprises a heavy chain variable region having a sequence at least 98% identical to SEQ ID NO: 33. In a further embodiment, the second monoclonal antibody comprises a light chain variable region having a sequence at least 95% identical to SEQ ID NO: 31. In a further embodiment, the second monoclonal antibody comprises a light chain variable region having a sequence at least 98% identical to SEQ ID NO: 31. In a further embodiment, the second monoclonal antibody has a heavy chain variable region having a sequence at least 95% identical to SEQ ID NO:33 and a light chain variable region having a sequence at least 95% identical to SEQ ID NO: 31. In a preferred embodiment, the second monoclonal antibody has a heavy chain variable region having a sequence at least 98% identical to SEQ ID NO: 33 and a light chain variable region having a sequence at least 98% identical to SEQ ID NO: 31.

As is known to the person skilled in the art, antibodies contain multiple regions including framework regions. Deletion or addition of amino acids in the framework regions is unlikely to affect the ability of the antibody to bind to its target. On the other hand, mutations in the CDRs are considerably more likely to affect the ability of an antibody to bind to a target. Thus, in certain embodiments of the invention, variant antibodies have CDRs which are identical to the CDRs of the 12A7 or 4B4 antibodies or have CDRs which vary in only a single amino acid substitution (preferably a conservative amino acid substitution). The first monoclonal antibody and/or the second monoclonal antibody may have framework regions which differ in sequence quite significantly from those described in SEQ ID NO: 27, 29, 31 or 33.

Optionally, where the first monoclonal antibody comprises a heavy chain variable region having a sequence at least 85%, 90%, 95%, 98%, 99% or 100% identical to SEQ ID NO: 29, the antibody further comprises at least one of 12A7 CDRH1, 12A7 CDRH2 or 12A7 CDRH3. It is understood by the person skilled in the art that, since target binding specificity is determined by the CDRs, an antibody comprising the CDRs of 12A7 may still bind to MCM5 even if the remainder of the antibody sequence is quite variable. For this reason where the first monoclonal antibody comprises at least one of 12A7 CDRH1, 12A7 CDRH2 or 12A7 CDRH3 the first monoclonal antibody preferably comprises a heavy chain variable region having a sequence at least 90% identical to SEQ ID NO: 29. In a more preferred embodiment the first monoclonal antibody of the invention comprises 12A7 CDRH1, 12A7 CDRH2 and 12A7 CDRH3 and comprises a heavy chain variable region having a sequence at least 95% identical to SEQ ID NO:29.

Optionally, where the second monoclonal antibody comprises a heavy chain variable region having a sequence at least 85%, 90%, 95%, 98%, 99% or 100% identical to SEQ ID NO: 33, the second monoclonal antibody further comprises at least one of 4B4 CDRH1, 4B4 CDRH2 or 4B4 CDRH3. It is understood by the person skilled in the art that, since target binding specificity is determined by the CDRs, an antibody comprising the CDRs of 4B4 may still bind to MCM5 even if the remainder of the antibody sequence is quite variable. For this reason, where the second monoclonal antibody comprises at least one of 4B4 CDRH1, 4B4 CDRH2 or 4B4 CDRH3 the second monoclonal antibody preferably comprises a heavy chain variable region having a sequence at least 90% identical to SEQ ID NO: 33. In a more preferred embodiment, the second monoclonal antibody comprises 4B4 CDRH1, 4B4 CDRH2 and 4B4 CDRH3 and comprises a heavy chain variable region having a sequence at least 95% identical to SEQ ID NO: 33.

Optionally, where the first monoclonal antibody comprises a light chain variable region having a sequence at least 85%, 90%, 95%, 98%, 99% or 100% identical to SEQ ID NO: 27, the first monoclonal antibody further comprises at least one of 12A7 CDRL1, 12A7 CDRL2 or 12A7 CDRL3. It is understood by the person skilled in the art that, since target binding specificity is determined by the CDRs, an antibody comprising the CDRs of 12A7 may still bind to MCM5 even if the remainder of the antibody sequence is quite variable. For this reason, where the first monoclonal antibody comprises at least one of 12A7 CDRL1, 12A7 CDRL2 or 12A7 CDRL3 the first monoclonal antibody preferably comprises a light chain variable region having a sequence at least 90% identical to SEQ ID NO: 27. In a more preferred embodiment, the first monoclonal antibody comprises 12A7 CDRL1, 12A7 CDRL2 and 12A7 CDRL3 and comprises a light chain variable region having a sequence at least 95% identical to SEQ ID NO: 27.

Optionally, where the second monoclonal antibody of the invention comprises a light chain variable region having a sequence at least 85%, 90%, 95%, 98%, 99% or 100% identical to SEQ ID NO: 31, the second monoclonal antibody further comprises at least one of 4B4 CDRL1, 4B4 CDRL2 or 4B4 CDRL3. It is understood by the person skilled in the art that, since target binding specificity is determined by the CDRs, an antibody comprising the CDRs of 4B4 may still bind to MCM5 even if the remainder of the antibody sequence is quite variable. For this reason, where the second monoclonal antibody comprises at least one of 4B4 CDRL1, 4B4 CDRL2 or 4B4 CDRL3 the second monoclonal antibody preferably comprises a heavy chain variable region having a sequence at least 90% identical to SEQ ID NO: 31. In a more preferred embodiment, the second monoclonal antibody comprises 4B4 CDRL1, 4B4 CDRL2 and 4B4 CDRL3 and comprises a heavy chain variable region having a sequence at least 95% identical to SEQ ID NO:31.

In a further embodiment of the invention, the first monoclonal antibody comprises:
(i) 12A7 CDRH1, 12A7 CDRH2 and 12A7 CDRH3;
(ii) a heavy chain variable region having a sequence at least 95% identical to SEQ ID NO: 29;
(iii) 12A7 CDRL1, 12A7 CDRL2 and 12A7 CDRL3; and
(iv) a light chain variable region having a sequence at least 95% identical to SEQ ID NO: 27.

In a further embodiment, the second monoclonal antibody comprises:
(i) 4B4 CDRH1, 4B4 CDRH2 and 4B4 CDRH3;
(ii) a heavy chain variable region having a sequence at least 95% identical to SEQ ID NO: 33;
(iii) 4B4 CDRL1, 4B4 CDRL2 and 4B4 CDRL3; and
(iv) a heavy chain variable region having a sequence at least 95% identical to SEQ ID NO: 31.

An antibody having a heavy chain variable sequence identical to SEQ ID NO: 29 and a light chain variable sequence identical to SEQ ID NO: 27 may be referred to as antibody 12A7. An antibody having a heavy chain variable sequence identical to SEQ ID NO: 33 and a light chain variable sequence identical to SEQ ID NO: 31 may be referred to as an antibody 4B4.

It is well within the knowledge of the person skilled in the art how to make variant antibodies which bind to MCM5. Such variant antibodies may comprise 1, 2, 3, 4, 5, 6, 7, 8, 9, 10, 15, 20, 25, 50 or 100 substitution or deletion mutations compared to SEQ ID NOs: 27, 29, 31 or 33. ‘Deletion’ variant antibodies may comprise the deletion of 1, 2, 3, 4, 5 or more amino acids or, in some cases, the deletion of entire regions of SEQ ID NOs: 27, 29, 31 or 33. ‘Substitution’ variants may comprise the replacement of 1, 2, 3, 4, 5 or more amino acids with the same number of new amino acids.

Preferably, the variant antibodies described herein comprise sequences differing from SEQ ID NOs: 27, 29, 31 or 33 by conservative amino acid substitutions (optionally only by conservative amino acid substitutions). The skilled person is well aware that such conservative substitutions are unlikely to alter the binding properties of an antibody.

In some embodiments, the ELISA assay or sandwich ELISA assay comprises the use of one or more of the monoclonal antibodies described herein. In one embodiment, the ELISA assay or sandwich ELISA assay comprises exposing the semen sample to a first monoclonal antibody described herein. In one embodiment, the ELISA assay or sandwich ELISA assay comprises exposing the semen sample to a second monoclonal antibody described herein. In one embodiment, the ELISA assay or sandwich ELISA assay comprises exposing the semen sample to first monoclonal antibody described herein and/or second monoclonal antibody described herein. In one embodiment, the ELISA assay or sandwich ELISA assay comprises exposing the semen sample to first monoclonal antibody described herein and second monoclonal antibody described herein. In some embodiments, the first monoclonal antibody and/or the second monoclonal antibody used in the ELISA assay or sandwich ELISA assay bind specifically to MCM5.

In some embodiments, the first monoclonal antibody used in the ELISA assay or sandwich ELISA assay binds to SEQ ID NO: 1. In some embodiments, the second monoclonal antibody used in the ELISA assay or sandwich ELISA assay binds to SEQ ID NO: 2. In some embodiments, the first monoclonal antibody used in the ELISA assay or sandwich ELISA assay binds to SEQ ID NO: 1 and the second monoclonal antibody used in the ELISA assay or sandwich ELISA assay binds to SEQ ID NO: 2.

In some embodiment, the first monoclonal antibody used in the ELISA assay or sandwich ELISA comprises at least one of the CDRs of 12A7 described herein. In some embodiments, the second monoclonal antibody used in the ELISA assay or sandwich ELISA assay comprises at least one of the CDRs of 4B4 described herein. In some embodiments, the first monoclonal antibody used in the ELISA assay or sandwich ELISA assay comprises at least one of the CDRs of 12A7 described herein and the second monoclonal antibody used in the ELISA assay or sandwich ELISA assay comprises at least one of the CDRs of 4B4 described herein.

In some embodiments, the first monoclonal antibody used in the ELISA assay or sandwich ELISA is a monoclonal antibody described herein which comprises at least one of the CDRs of 12A7. In some embodiments, the second monoclonal antibody used in the ELISA assay or sandwich ELISA assay is a monoclonal antibody described herein which comprises at least one of the CDRs of 4B4. In some embodiments, the first monoclonal antibody used in the ELISA assay or sandwich ELISA assay is a monoclonal antibody described herein which comprises at least one of the CDRs of 12A7 and the second monoclonal antibody used in the ELISA assay or sandwich ELISA assay is a monoclonal antibody described herein which comprises at least one of the CDRs of 4B4.

In some embodiments, the first monoclonal antibody used in the ELISA assay or sandwich ELISA is a monoclonal antibody described herein which comprises at least one of the CDRs of 12A7 and binds to SEQ ID NO: 1. In some embodiments, the second monoclonal antibody used in the ELISA assay or sandwich ELISA assay is a monoclonal antibody described herein which comprises at least one of the CDRs of 4B4 and binds to SEQ ID NO: 2. In some embodiments, the first monoclonal antibody used in the ELISA assay or sandwich ELISA assay is a monoclonal antibody described herein which comprises at least one of the CDRs of 12A7 and binds to SEQ ID NO: 1 and the second monoclonal antibody used in the ELISA assay or sandwich ELISA assay is a monoclonal antibody which comprises at least one of the CDRs of 4B4 and binds to SEQ ID NO: 2.

In some embodiments, the first monoclonal antibody used in the ELISA assay or sandwich ELISA is 12A7. In some embodiments, the second monoclonal antibody used in the ELISA assay or sandwich ELISA assay is 4B4. In some embodiments, the first monoclonal antibody used in the ELISA assay or sandwich ELISA assay is 12A7 and the second monoclonal antibody used in the ELISA assay or sandwich ELISA assay is 4B4.

Uses

The present invention also provides uses of various components disclosed herein. In one embodiment, the present invention provides the use of a lysis buffer of the invention in a method of detecting the presence or absence of prostate cancer in a subject. In one embodiment, the present invention provides the use of a first monoclonal antibody of the invention and/or a second monoclonal antibody of the invention in a method of detecting the presence or absence of prostate cancer in a subject.

Kits

The present invention also provides a use of a kit in a method of detecting the presence or absence of prostate cancer in a subject, wherein the kit comprises:

(a) a lysis buffer as defined in any one of the preceding claims;

(b) a first monoclonal antibody as defined in any one of the preceding claims; and/or

(c) a second monoclonal antibody as defined in any one of the preceding claims.

The present invention also provides a kit comprising:

(a) a lysis buffer as defined in any one of the preceding claims;

(b) a first monoclonal antibody as defined in any one of the preceding claims; and/or

(c) a second monoclonal antibody as defined in any one of the preceding claims; and

(d) instructions for use of the lysis buffer and/or the first monoclonal antibody and/or the second monoclonal antibody in a method of detecting the presence or absence of prostate cancer in a subject.

TABLE 1
Sequences

| SEQ ID NO | Nucleotide/Polypeptide |
|---|---|
| 1 | WDETKGE (epitope to which antibody 12A7 binds) |
| 2 | DDRVAIH (epitope to which antibody 4B4 binds |
| 3 | 12A7 light chain CDR 1 polypeptide sequence |
| 4 | 12A7 light chain CDR 1 nucleotide sequence |
| 5 | 12A7 light chain CDR 2 polypeptide sequence |
| 6 | 12A7 light chain CDR 2 nucleotide sequence |
| 7 | 12A7 light chain CDR 3 polypeptide sequence |
| 8 | 12A7 light chain CDR 3 nucleotide sequence |
| 9 | 12A7 heavy chain CDR 1 polypeptide sequence |
| 10 | 12A7 heavy chain CDR 1 nucleotide sequence |
| 11 | 12A7 heavy chain CDR 2 polypeptide sequence |
| 12 | 12A7 heavy chain CDR 2 nucleotide sequence |
| 13 | 12A7 heavy chain CDR 3 polypeptide sequence |
| 14 | 12A7 heavy chain CDR 3 nucleotide sequence |
| 15 | 4B4 light chain CDR 1 polypeptide sequence |
| 16 | 4B4 light chain CDR 1 nucleotide sequence |
| 17 | 4B4 light chain CDR 2 polypeptide sequence |
| 18 | 4B4 light chain CDR 2 nucleotide sequence |
| 19 | 4B4 light chain CDR 3 polypeptide sequence |
| 20 | 4B4 light chain CDR 3 nucleotide sequence |
| 21 | 4B4 heavy chain CDR 1 polypeptide sequence |
| 22 | 4B4 heavy chain CDR 1 nucleotide sequence |
| 23 | 4B4 heavy chain CDR 2 polypeptide sequence |
| 24 | 4B4 heavy chain CDR 2 nucleotide sequence |
| 25 | 4B4 heavy chain CDR 3 polypeptide sequence |
| 26 | 4B4 heavy chain CDR 3 nucleotide sequence |
| 27 | 12A7 full light chain variable region sequence |
| | (polypeptide) |
| 28 | 12A7 full light chain variable region sequence |
| | (nucleotide) |
| 29 | 12A7 full heavy chain variable region sequence |
| | (polypeptide) |
| 30 | 12A7 full heavy chain variable region sequence |
| | (nucleotide) |
| 31 | 4B4 full light chain variable region sequence |
| | (polypeptide) |
| 32 | 4B4 full light chain variable region sequence |
| | (nucleotide) variable region |
| 33 | 4B4 full heavy chain variable region sequence |
| | (polypeptide) |
| 34 | 4B4 full heavy chain variable region sequence |
| | (nucleotide) |
| 35 | MCM5 polypeptide sequence |
| 36 | MCM5 polynucleotide sequence |

### Example

Study Population

Patients were enrolled into the study at 4 UK sites between March and October 2018, ethical approval was obtained from North East—Newcastle & North Tyneside 1 Research Ethics Committee and informed consent obtained from all patients prior to the collection of urine and semen samples.

Sample Processing-Urine

A minimum of 10 mL urine was collected from each patient following a digital rectal examination, urine was agitated to ensure a homogenous mix and between 10 and 50 mL was transferred into a clean centrifuge tube. Samples were centrifuged at room temperature at 1500 g for 5 minutes. Supernatent was discarded to waste taking care not to disturb the cell sediment pellet, tubes were placed upside down on absorbent paper. Cell sediment pellets were resuspended in an appropriate volume of Lysis buffer (10 μL per mL of urine) and incubated at room temperature for 1 hr before being stored at less than −20° C.

Sample Processing-Semen

Patients were asked to provide samples no more than 48 hours prior to their appointment and hand to the research nurse at their appointment. Samples were then transferred to the laboratory and centrifuged at 1500 g for 5 minutes. Following centrifugation the supernatant was removed and placed in a fresh tube before being stored at −20° C. or below. The remaining cell sediment pellet was then resuspended in 500 μL of lysis buffer and incubated at room temperature for 3 minutes to 1 hour to allow lysis to occur before being stored at −20° C. or below until testing.

MCM5 ELISA

Patient lysates were subjected to an MCM5 ELISA test as per manufacturers instructions. Briefly 100 μL of lysate was added to the MCM5 ELISA micro-titre plate (samples and controls were run in duplicates) and incubated for 60 minutes at room temperature on a plate shaker (700 RPM), following incubation wells were washed 6 times with 350 μL of 1× wash buffer using an automated plate washer. 100 μL of MCM5-HRP conjugated antibody was added to each well and incubated at room temperature for 30 minutes prior to being washed 6 times with 350 μl of 1× wash buffer as above. 100 μL of TMB was added to each well and incubated for 30 minutes in the dark prior to the addition of a stop solution (0.5 M H₂SO₄). Optical density (OD) was measured at 450 nm and 630 nm (reference wavelength). Concentrations of MCM5 were calculated using a serial dilution standard curve of a known recombinant MCM5 control (1.5 mg/ml) with a negative control (Lysis Buffer).

Results—Urine

Voided urine samples were collected immediately following a Digital Rectal Examination by the urologist (DRE), samples were collected from a total of 146 eligible subjects, ranging in age from 48-80 years old. 70 subjects were subsequently diagnosed as having a prostate cancer by routine clinical investigations based on prostatic biopsy.

The remaining 76 subjects were found to have no prostate cancer as defined by a negative biopsy result (no tumour cells were seen in a biopsy sample). Table 1 shows the sensitivity, specificity, Positive Predictive Value (PPV) and Negative Predictive Value (NPV) of ADXPROSTATE in post-DRE urine when calculated using the mean of duplicate wells. Calculations using single well results may result in a change in performance characteristics.

TABLE 1
ADXPROSTATE performance, when compared
to the reference tests (DRE, MRI and biopsy).
ADXPROSTATE Results: Sensitivity,
Specificity, PPV and NPV
in suspected prostate cancer patients

| | Sensitivity % | Specificity % | PPV % | NPV % |
|---|---|---|---|---|
| | (TP/ | (TN/ | (TP/ | (TN/ |
| | (TP + FN)) | (TN + FP)) | (TP + FP)) | (TN + FN)) |
| Urine | (95% CI) | (95% CI) | (95% CI) | (95% CI) |
| ADXPROSTATE | 32.9 | 63.2 | 45.1 | 50.5 |
| (cut-off ≥ 3 pg/ml) | 23/70 | 48/76 | 23/51 | 48/95 |
| | 22.1% to | 51.3% to | 34.5% to | 44.6% to |
| | 45.1% | 73.9% | 56.2% | 56.4% |

Results—Semen

Semen samples were collected from a total of 42 eligible subjects, ranging in age from 48-72. 23 subjects were subsequently diagnosed as having a prostate cancer by routine clinical investigations based on prostatic biopsy.

The remaining 19 subjects were found to have no prostate cancer as defined by a negative biopsy result (no tumour cells were seen in a biopsy sample), or the urologist determining that there was no need to biopsy based upon DRE, PSA and imaging. Table 2 shows the sensitivity, specificity, Positive Predictive Value (PPV) and Negative Predictive Value (NPV) of ADXPROSTATE in semen when calculated using the mean of duplicate wells. Calculations using single well results may result in a change in performance characteristics.

TABLE 2
ADXPROSTATE performance in semen-pellet, when
compared to reference tests (DRE, MRI and biopsy).
ADXPROSTATE Results: Sensitivity, Specificity, PPV
and NPV in suspected prostate cancer patients

| | Sensitivity % | Specificity % | PPV % | NPV % |
|---|---|---|---|---|
| | (TP/ | (TN/ | (TP/ | (TN/ |
| | (TP + FN)) | (TN + FP)) | (TP + FP)) | (TN + FN)) |
| Semen | (95% CI) | (95% CI) | (95% CI) | (95% CI) |
| ADXPROSTATE | 61 | 79 | 78 | 63 |
| (cut-off ≥ 65 pg/ml) | 14/23 | 15/19 | 14/18 | 15/24 |
| | 38.5% to | 54.4% to | 58.0% to | 48.8% to |
| | 80.3% | 94.0% | 89.9% | 74.5% |

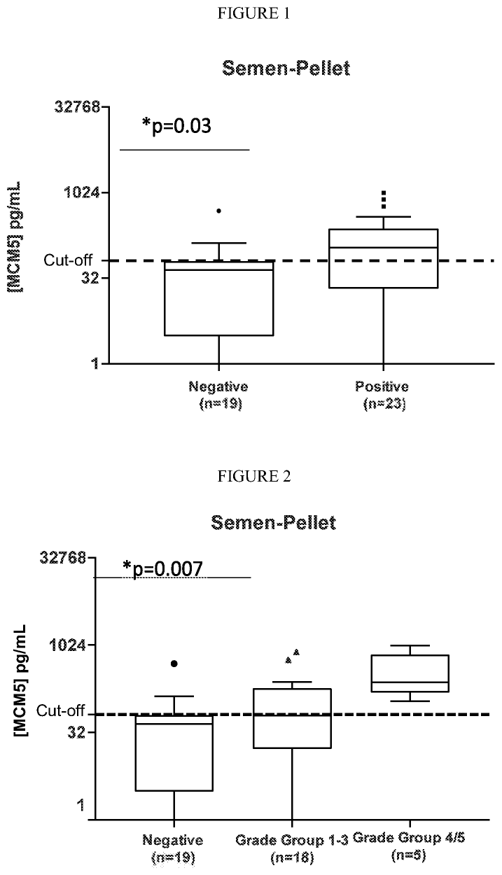

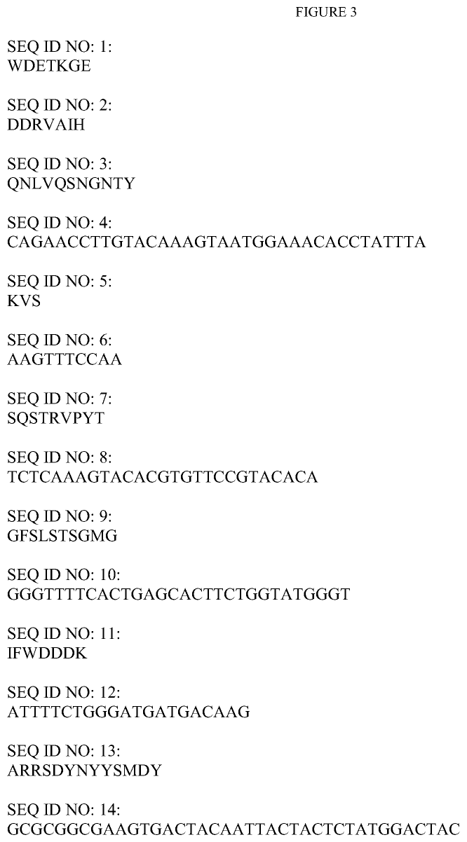

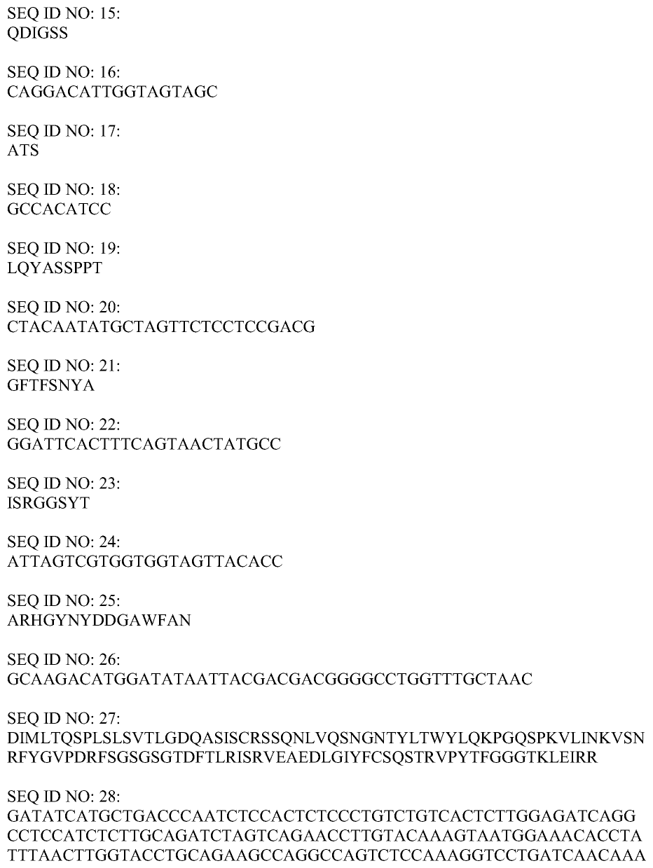

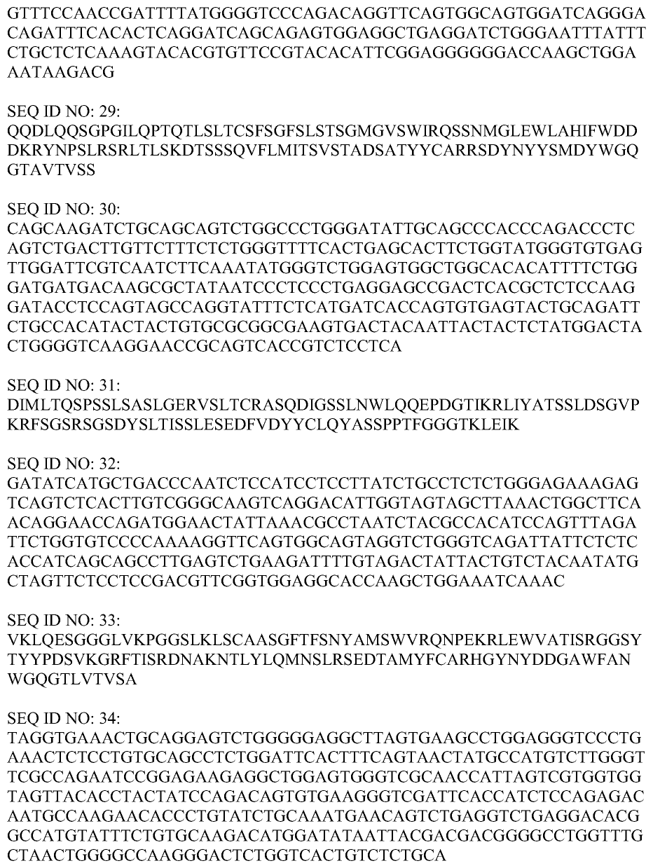

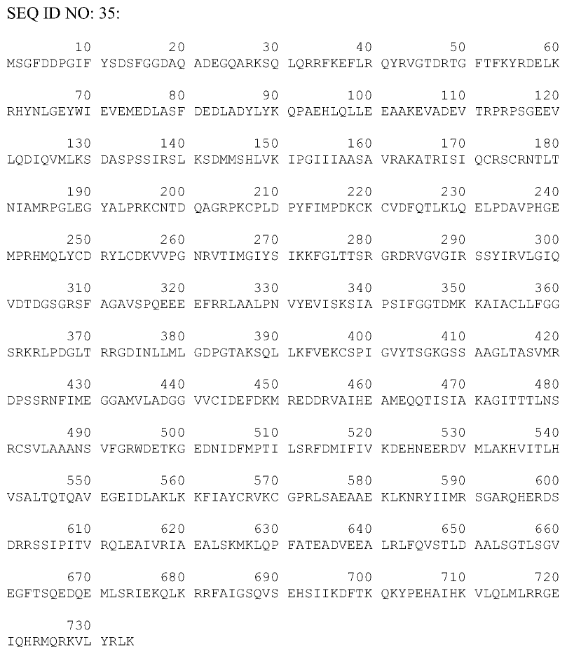

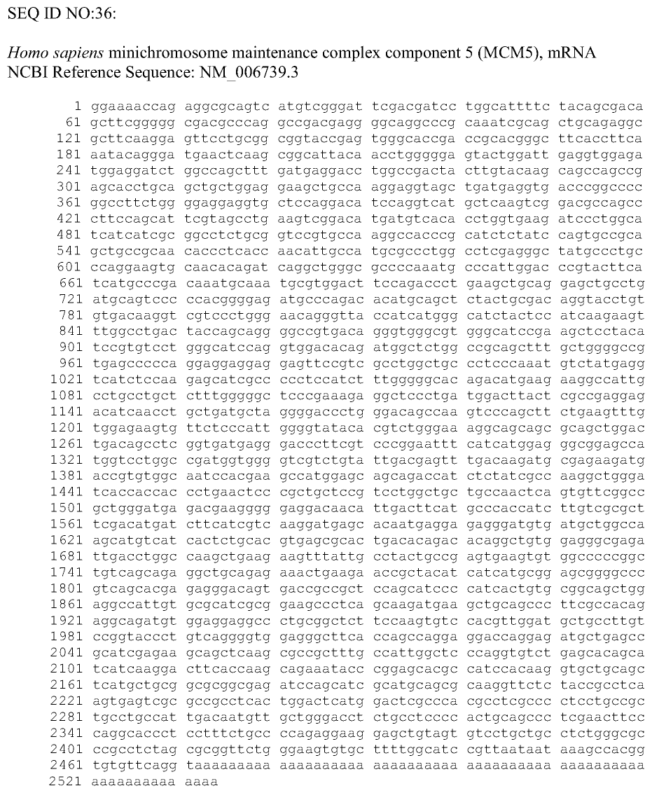

Levels of MCM5 as determined using ADXPROSTATE were significantly higher in the semen-pellet of patients with prostate cancer (FIG. 1, p=0.03). Importantly in all 5 patients with Gleason Grade Group 4 and 5, MCM5 levels were significantly higher than the cut-off (FIG. 2, p=0.007).

Further aspects of the invention:

1. A method for detecting the presence or absence of a prostate cancer in a subject, the method comprising steps of:
providing a semen sample obtained from the subject; and
detecting at least one biomarker or determining the concentration of at least one biomarker.

2. The method of aspect 1, further comprising a step of treating the semen sample to release the at least one biomarker from cells in the semen sample.

3. The method of aspect 1 or 2, wherein the at least one biomarker comprises or consists of an MCM protein.

4. The method of aspect 3, wherein the MCM protein is MCM5.

5. The method of any one of the preceding aspects, wherein the semen sample is obtained after a period of abstinence.

6. The method of any one of the preceding aspects, wherein detecting the at least one biomarker comprises determining whether the concentration of the biomarker is higher than a pre-defined cut-off.

7. The method of aspect 6, wherein the pre-defined cut-off is between 40 pg/mL and 100 pg/mL, between 50 pg/mL and 75 pg/mL, between 60 pg/mL and 70 pg/mL, or around 65 pg/mL.

8. The method of aspect 6 or 7, wherein prostate cancer is likely to be present if the concentration of the biomarker is higher than the pre-defined cut-off.

9. The method of any one of aspects 6-8, wherein the method is a method for diagnosing prostate cancer, and the method further comprises a step of diagnosing the subject as having prostate cancer if the concentration of the biomarker is higher than the pre-defined cut-off.

10. The method of any one of the preceding aspects, wherein the concentration of the at least one biomarker is compared to a reference, and prostate cancer is likely to be present if the concentration of the at least one biomarker is abnormal compared to or higher than the reference.

11. The method of aspect 10, wherein the method is a method for diagnosing prostate cancer, and the method further comprises a step of diagnosing the subject as having prostate cancer if the concentration of the at least one biomarker is higher than the reference.

12. The method of any one of the preceding aspects, wherein determining the concentration of the at least one biomarker or determining whether the concentration of the at least one biomarker is higher than a pre-defined cut-off is carried out using an ELISA assay.

13. The method of any one of the preceding aspects, wherein the method has a sensitivity of greater than 40%, greater than 50%, greater than 60%, between 40% and 100%, between 50% and 100%, between 60% and 100%, or around 61%.

14. The method of any one of the preceding aspects, wherein the method has a sensitivity of greater than 40%, greater than 50%, greater than 60%, between 40% and 100%, between 50% and 100%, between 60% and 100%, or around 61% using a pre-defined cut-off of 65 pg/mL.

15. The method of any one of the preceding aspects, wherein the method has a specificity of greater than 70%, greater than 75%, between 70% and 100%, between 75% and 100%, or around 79%.

16. The method of any one of the preceding aspects, wherein the method has a specificity of greater than 70%, greater than 75%, between 70% and 100%, between 75% and 100%, or around 79% using a pre-defined cut-off of 65 pg/mL.

17. The method of any one of the preceding aspects, wherein the method has a positive predictive value (PPV) of greater than 60%, greater than 70%, greater than 75%, between 60% and 100%, between 70% and 100%, between 75% and 100%, or around 78%.

18. The method of any one of the preceding aspects, wherein the method has a positive predictive value (PPV) of greater than 60%, greater than 70%, greater than 75%, between 60% and 100%, between 70% and 100%, between 75% and 100%, or around 78% using a pre-defined cut-off of 65 pg/mL.

19. The method of any one of the preceding aspects, wherein the method has a negative predictive value (NPV) of greater than 55%, greater than 60%, between 55% and 100%, between 60% and 100%, or around 63%.

20. The method of any one of the preceding aspects, wherein the method has a negative predictive value (NPV) of greater than 55%, greater than 60%, between 55% and 100%, between 60% and 100%, or around 63% using a pre-defined cut-off of 65 pg/mL.

21. The method of any one of the preceding aspects, wherein the method has greater sensitivity, specificity, NPV and/or PPV than an equivalent method carried out using a urine sample.

22. The method of aspect 21, wherein the equivalent method carried out using a urine sample uses a cut-off of 3 pg/mL.

23. The method of any one of aspects 2-22, wherein the step of treating the semen sample to release the at least one biomarker comprises exposing the semen sample to a lysis buffer capable of releasing the at least one biomarker from cells in the semen sample.

24. The method of aspect 23, wherein exposing the semen sample to a lysis buffer capable of releasing the at least one biomarker from cells in the semen sample is carried out on a pellet of the cells in the semen sample obtained by centrifuging the semen sample.

25. The method of aspect 23 or 24, wherein exposing the semen sample to a lysis buffer capable of releasing the at least one biomarker from cells in the semen sample comprises:

passing the non-invasive sample through a filter for capturing cells, such that cells are captured in the filter;

passing a lysis buffer through the filter, such that the captured cells are exposed to the lysis buffer; and/or

incubating the filter for a period of time, such that the lysis buffer causes the cells to release the at least one biomarker.

26. The method of any one of aspects 23-25, wherein the lysis buffer is capable of releasing an MCM protein from cells in the semen sample, optionally wherein the cells are epithelial cells.

27. The method of aspect 26, wherein the lysis buffer is capable of releasing MCM5 from cells in the semen sample, optionally wherein the cells are epithelial cells.

28. The method of any one of aspects 23-27, wherein the lysis buffer is capable of releasing MCM5 from cells in the semen sample and does not denature the MCM5 protein, optionally wherein the cells are epithelial cells.

29. The method of any one of aspects 23-28, wherein the lysis buffer does not denature an antibody.

30. The method of any one of aspects 23-29, wherein the lysis buffer comprises a detergent.

31. The method of aspect 30, wherein the detergent comprises Triton X-100.

32. The method of aspect 31, wherein the detergent comprises Triton X-100 at a concentration of between 0.01% and 25%, between 0.01% and 10%, between 0.05% and 5%, between 0.1% and 2%, between 0.5% and 2%, between 0.75% and 1.25%, or about 1%.

33. The method of any one of aspects 30-32, wherein the detergent comprises or consists of sodium deoxycholate.

34. The method of aspect 33, wherein the detergent comprises or consists of sodium deoxycholate at a concentration between 0.1% and 20%, between 0.1 and 10%, between 0.1 and 5%, between 0.5% and 5%, between 0.5% and 2.5%, between 0.75% and 2.5%, between 0.75% and 1.25%, or about 1%.

35. The method of any one of aspects 30-34, wherein the detergent comprises or consists of sodium dodecyl sulphate (SDS).

36. The method of aspect 35, wherein the detergent comprises or consists of sodium dodecyl sulphate (SDS) at a concentration of between 0.001% and 10%, between 0.01% and 5%, between 0.05% and 5%, between 0.01% and 1%, between 0.05% and 1%, between 0.05% and 0.5%, between 0.075% and 0.25%, or about 0.1%.

37. The method of any one of aspects 30-36, wherein the detergent consists of Triton X-100, sodium deoxycholate, and sodium dodecyl sulphate (SDS).

38. The method of aspect 37, wherein the detergent consists of between 0.5% and 2% of Triton X-100, between 0.5% and 2% of sodium deoxycholate, and between 0.05% and 0.5% of sodium dodecyl sulphate (SDS).

39. The method of any one of aspects 23-38, wherein the lysis buffer comprises a buffer component.

40. The method of aspect 39, wherein the buffer component has a pH of between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6; and/or maintains the pH of the lysis buffer at between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6.

41. The method of aspect 39 or 40, wherein the buffer component comprises or consists of Tris.

42. The method of aspect 41, wherein the buffer component comprises or consists of Tris at a concentration greater than 1 mM, between 1 mM and 350 mM, between 5 and 200 mM, between 5 and 100 mM, between 5 and 50 mM, between 5 mM and 40 mM, between 5 mM and 35 mM, between 10 mM and 35 mM, between 15 mM and 35 mM, between 15 mM and 30 mM, between 20 mM and 30 mM, or about 25 mM.

43. The method of any one of aspects 39-42, wherein the buffer component consists of Tris at a concentration of between 15 mM and 35 mM.

44. The method of any one of aspects 23 to 43, wherein the lysis buffer comprises a salt.

45. The method of aspect 44, wherein the salt is sodium chloride.

46. The method of aspect 45, wherein the lysis buffer comprises sodium chloride at a concentration between 10 mM and 350 mM, between 20 mM and 300 mM, between 50 mM and 250 mM, between 100 mM and 250, between 100 mM and 200 mM, between 125 mM and 175 mM, or about 150 mM.

47. The method of aspect 46, wherein the lysis buffer comprises sodium chloride at a concentration of between 100 mM and 200 mM.

48. The method of any one of aspects 23 to 47, wherein the lysis buffer comprises:

(i) between 1 mM and 100 mM Tris;

(ii) between 50 mM and 300 mM sodium chloride;

(iii) between 0.1 and 5% sodium deoxycholate;

(iv) between 0.01 and 1% sodium dodecyl sulphate; and/or

(v) between 0.1 and 5% Triton-X100.

49. The method of aspect 48, wherein the lysis buffer comprises:

(i) between 10 mM and 40 mM Tris;

(ii) between 100 mM and 200 mM sodium chloride;

(iii) between 0.5 and 2% sodium deoxycholate;

(iv) between 0.05 and 0.5% sodium dodecyl sulphate; and/or

(v) between 0.5 and 2% Triton-X100.

50. The method of aspect 49, wherein the lysis buffer comprises:

(i) about 25 mM Tris;

(ii) about 150 mM sodium chloride;

(iii) about 1% sodium deoxycholate;

(iv) about 0.1% sodium dodecyl sulphate; and/or

(v) about 1% Triton-X100.

51. The method of any one of aspects 2-50, wherein the cells which release the at least one biomarker are epithelial cells, optionally epithelial cells which have been shed from the lining of the prostate gland of the subject.

52. The method of any one of the preceding aspects, wherein the step of detecting the at least one biomarker or determining the concentration of the at least one biomarker comprises:

exposing the semen sample to a first monoclonal antibody and/or a second monoclonal antibody; and

detecting the at least one biomarker bound to the first monoclonal antibody and/or the second monoclonal antibody or determining the concentration of the at least one biomarker bound to the first monoclonal antibody and/or the second monoclonal antibody.

53. The method of aspect 52, wherein the first monoclonal antibody and the second monoclonal antibody bind to MCM5.

54. The method of aspect 53, wherein the first monoclonal antibody is an antibody which:

(i) binds to a polypeptide having an amino acid sequence of SEQ ID NO: 1;

(ii) comprises at least one Complementary Determining Region (CDR) selected from the group consisting of:
(a) 12A7 CDRH1 which has a sequence of SEQ ID NO: 9 or a sequence that differs from SEQ ID NO:9 by a single amino acid substitution;
(b) 12A7 CDRH2 which has a sequence of SEQ ID NO: 11 or a sequence that differs from SEQ ID NO:11 by a single amino acid substitution;
(c) 12A7 CDRH3 which has a sequence of SEQ ID NO: 13 or a sequence that differs from SEQ ID NO:13 by a single amino acid substitution;
(d) 12A7 CDRL1 which has a sequence of SEQ ID NO: 3 or a sequence that differs from SEQ ID NO:3 by a single amino acid substitution;
(e) 12A7 CDRL2 which has a sequence of SEQ ID NO: 5 or a sequence that differs from SEQ ID NO:5 by a single amino acid substitution; and
(f) 12A7 CDRL3 which has a sequence of SEQ ID NO: 7 or a sequence that differs from SEQ ID NO:7 by a single amino acid substitution;

(iii) comprises a heavy chain variable region having a sequence at least 85%, 90%, 95%, 98%, 99%, or 100% identical to SEQ ID NO: 29;

(iv) comprises a light chain variable region sequence having a sequence at least 85%, 90%, 95%, 98%, 99%, or 100% identical to SEQ ID NO: 27; or

(v) competes with the antibody of (i), (ii), (iii), or (iv).

55. The method of aspect 53 or 54, wherein the second monoclonal antibody is an antibody which:

(i) binds to a polypeptide having an amino acid sequence of SEQ ID NO: 2;

(ii) comprises at least one Complementary Determining Region (CDR) selected from the group consisting of:
(a) 4B4 CDRH1 which has a sequence of SEQ ID NO: 21 or a sequence that differs from SEQ ID NO:21 by a single amino acid substitution;
(b) 4B4 CDRH2 which has a sequence of SEQ ID NO: 23 or a sequence that differs from SEQ ID NO:23 by a single amino acid substitution;
(c) 4B4 CDRH3 which has a sequence of SEQ ID NO: 25 or a sequence that differs from SEQ ID NO:25 by a single amino acid substitution;
(d) 4B4 CDRL1 which has a sequence of SEQ ID NO: 15 or a sequence that differs from SEQ ID NO:15 by a single amino acid substitution;
(e) 4B4 CDRL2 which has a sequence of SEQ ID NO: 17 or a sequence that differs from SEQ ID NO:17 by a single amino acid substitution; and
(f) 4B4 CDRL3 which has a sequence of SEQ ID NO: 19 or a sequence that differs from SEQ ID NO:19 by a single amino acid substitution;

(iii) comprises a heavy chain variable region having a sequence at least 85%, 90%, 95%, 98%, 99%, or 100% identical to SEQ ID NO: 33;

(iv) comprises a light chain variable region sequence having a sequence at least 85%, 90%, 95%, 98%, 99%, or 100% identical to SEQ ID NO: 31; or

(v) competes with the antibody of (i), (ii), (iii), or (iv).

56. The method of any one of aspects 52-55, wherein the first monoclonal antibody and/or second monoclonal antibody has an affinity for MCM5 in the range of 0.001-1 nM.

57. The method of any one of aspects 52-56, wherein the first monoclonal antibody and/or the second monoclonal antibody is a Fab′2, a F′(ab)₂, an Fv, a single chain antibody or a diabody.

58. The method of any one of aspects 52-57, wherein the first monoclonal antibody comprises 12A7 CDRH1, 12A7 CDRH2, and 12A7 CDRH3.

59. The method of any one of aspects 52-58, wherein the first monoclonal antibody comprises 12A7 CDRL1, 12A7 CDRL2, and 12A7 CDRL3.

60. The method of any one of aspects 52-59, wherein the first monoclonal antibody comprises a 12A7 CDRH1 which has a sequence of SEQ ID NO: 9, a 12A7 CDRH2 which has a sequence of SEQ ID NO: 11, and a 12A7 CDRH3 which has a sequence of SEQ ID NO: 13.

61. The method of any one of aspects 52-60, wherein the first monoclonal antibody comprises a 12A7 CDRL1 which has a sequence of SEQ ID NO: 3, a 12A7 CDRL2 which has a sequence of SEQ ID NO: 5, and a 12A7 CDRL3 which has a sequence of SEQ ID NO: 7.

62. The method of any one of aspects 52-61, wherein the second monoclonal antibody comprises 4B4 CDRH1, 4B4 CDRH2, and 4B4 CDRH3.

63. The method of any one of aspects 52-62, wherein the second monoclonal antibody comprises 4B4 CDRL1, 4B4 CDRL2 and 4B4 CDRL3.

64. The method of any one of aspects 52-63, wherein the second monoclonal antibody comprises a 4B4 CDRH1 which has a sequence of SEQ ID NO: 21, a 4B4 CDRH2 which has a sequence of SEQ ID NO: 23, and a 4B4 CDRH3 which has a sequence of SEQ ID NO: 25.

65. The method of any one of aspects 52-64, wherein the second monoclonal antibody has a 4B4 CDRL1 which has a sequence of SEQ ID NO: 15, a 4B4 CDRL2 which has a sequence of SEQ ID NO: 17, and a 4B4 CDRL3 which has a sequence of SEQ ID NO: 19.

66. The method of any one of aspects 52-65 wherein the first monoclonal antibody comprises a heavy chain variable region having a sequence at least 95% identical to SEQ ID NO: 29.

67. The method of any one of aspects 52-66, wherein the first monoclonal antibody comprises a heavy chain variable region having a sequence at least 98% identical to SEQ ID NO: 29.

68. The method of any one of aspects 52-67, wherein the first monoclonal antibody comprises a light chain variable region having a sequence at least 95% identical to SEQ ID NO: 27.

69. The method of any one of aspects 52-68, wherein the first monoclonal antibody comprises a light chain variable region having a sequence at least 98% identical to SEQ ID NO: 27.

70. The method of any one of aspects 52-69, wherein the second monoclonal antibody comprises a heavy chain variable region having a sequence at least 95% identical to SEQ ID NO: 33.

71. The method of any one of aspects 52-70, wherein the second monoclonal antibody comprises a heavy chain variable region having a sequence at least 98% identical to SEQ ID NO: 33.

72. The method of any one of aspects 52-71, wherein the second monoclonal antibody comprises a light chain variable region having a sequence at least 95% identical to SEQ ID NO: 31.

73. The method of any one of aspects 52-72, wherein the second monoclonal antibody comprises a light chain variable region having a sequence at least 98% identical to SEQ ID NO: 31.

74. Use of a lysis buffer as defined in any one of the preceding aspects in a method of detecting the presence of absence of prostate cancer in a subject.

75. Use of a first monoclonal antibody as defined in any one of the preceding aspects and/or a second monoclonal antibody as defined in any one of the preceding aspects in a method of detecting the presence or absence of prostate cancer in a subject.

76. Use of a kit in a method of detecting the presence or absence of prostate cancer in a subject, wherein the kit comprises:

(a) a lysis buffer as defined in any one of the preceding aspects;

(b) a first monoclonal antibody as defined in any one of the preceding aspects; and/or

(c) a second monoclonal antibody as defined in any one of the preceding aspects.

77. A kit comprising:

(a) a lysis buffer as defined in any one of the preceding aspects;

(b) a first monoclonal antibody as defined in any one of the preceding aspects; and/or

(c) a second monoclonal antibody as defined in any one of the preceding aspects; and

(d) instructions for use of the lysis buffer and/or the first monoclonal antibody and/or the second monoclonal antibody in a method of detecting the presence or absence of prostate cancer in a subject.

78. The method, use or kit of any one of the preceding aspects, wherein the prostate cancer is grade 4/5.

79. The method, use or kit of any one of aspects 1-78, wherein the prostate cancer is grade 1-3.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A method for detecting the presence or absence of a prostate cancer in a subject, the method comprising steps of:
providing a semen sample obtained from the subject; and
detecting at least one biomarker or determining the concentration of at least one biomarker.

2. The method of claim 1, further comprising a step of treating the semen sample to release the at least one biomarker from cells in the semen sample.

3. The method of claim 1 or 2, wherein the at least one biomarker comprises or consists of an MCM protein.

4. The method of claim 3, wherein the MCM protein is MCM5.

5. The method of any one of the preceding claims, wherein the semen sample is obtained after a period of abstinence.

6. The method of any one of the preceding claims, wherein:
detecting the at least one biomarker comprises determining whether the concentration of the biomarker is higher than a pre-defined cut-off.

7. The method of claim 6, wherein:
(a) the pre-defined cut-off is between 40 pg/mL and 100 pg/mL, between 50 pg/mL and 75 pg/mL, between 60 pg/mL and 70 pg/mL, or around 65 pg/mL;
(b) prostate cancer is likely to be present if the concentration of the biomarker is higher than the pre-defined cut-off; and/or
(c) the method is a method for diagnosing prostate cancer, and the method further comprises a step of diagnosing the subject as having prostate cancer if the concentration of the biomarker is higher than the pre-defined cut-off.

8. The method of any one of the preceding claims, wherein the concentration of the at least one biomarker is compared to a reference, and prostate cancer is likely to be present if the concentration of the at least one biomarker is abnormal compared to or higher than the reference, optionally wherein the method is a method for diagnosing prostate cancer, and the method further comprises a step of diagnosing the subject as having prostate cancer if the concentration of the at least one biomarker is higher than the reference.

9. The method of any one of the preceding claims, wherein:
(a) determining the concentration of the at least one biomarker or determining whether the concentration of the at least one biomarker is higher than a pre-defined cut-off is carried out using an ELISA assay;
(b) the method has a sensitivity of greater than 40%, greater than 50%, greater than 60%, between 40% and 100%, between 50% and 100%, between 60% and 100%, or around 61%;
(c) the method has a sensitivity of greater than 40%, greater than 50%, greater than 60%, between 40% and 100%, between 50% and 100%, between 60% and 100%, or around 61% using a pre-defined cut-off of 65 pg/mL;
(d) the method has a specificity of greater than 70%, greater than 75%, between 70% and 100%, between 75% and 100%, or around 79%;
(e) the method has a specificity of greater than 70%, greater than 75%, between 70% and 100%, between 75% and 100%, or around 79% using a pre-defined cut-off of 65 pg/mL;
(f) the method has a positive predictive value (PPV) of greater than 60%, greater than 70%, greater than 75%, between 60% and 100%, between 70% and 100%, between 75% and 100%, or around 78%;
(g) the method has a positive predictive value (PPV) of greater than 60%, greater than 70%, greater than 75%, between 60% and 100%, between 70% and 100%, between 75% and 100%, or around 78% using a pre-defined cut-off of 65 pg/mL;
(h) the method has a negative predictive value (NPV) of greater than 55%, greater than 60%, between 55% and 100%, between 60% and 100%, or around 63%;
(i) the method has a negative predictive value (NPV) of greater than 55%, greater than 60%, between 55% and 100%, between 60% and 100%, or around 63% using a pre-defined cut-off of 65 pg/mL; and/or
(j) the method has greater sensitivity, specificity, NPV and/or PPV than an equivalent method carried out using a urine sample, optionally wherein the equivalent method carried out using a urine sample uses a cut-off of 3 pg/mL.

10. The method of any one of claims 2-9, wherein the step of treating the semen sample to release the at least one biomarker comprises exposing the semen sample to a lysis buffer capable of releasing the at least one biomarker from cells in the semen sample, optionally wherein:
(a) exposing the semen sample to a lysis buffer capable of releasing the at least one biomarker from cells in the semen sample is carried out on a pellet of the cells in the semen sample obtained by centrifuging the semen sample;
(b) exposing the semen sample to a lysis buffer capable of releasing the at least one biomarker from cells in the semen sample comprises:
passing the non-invasive sample through a filter for capturing cells, such that cells are captured in the filter;
passing a lysis buffer through the filter, such that the captured cells are exposed to the lysis buffer; and/or
incubating the filter for a period of time, such that the lysis buffer causes the cells to release the at least one biomarker;
(c) the lysis buffer is capable of releasing an MCM protein from cells in the semen sample, optionally wherein the lysis buffer is capable of releasing MCM5 from cells in the semen sample;
(d) the lysis buffer is capable of releasing an MCM protein from epithelial cells in the semen sample, optionally wherein the lysis buffer is capable of releasing MCM5 from epithelial cells in the semen sample;
(e) the lysis buffer is capable of releasing MCM5 from cells in the semen sample and does not denature the MCM5 protein;
(f) the lysis buffer is capable of releasing MCM5 from epithelial cells in the semen sample and does not denature the MCM5 protein; and/or
(g) the lysis buffer does not denature an antibody.

11. The method of claim 10, wherein:
(a) the lysis buffer comprises a detergent;
(b) the lysis buffer comprises a detergent which comprises Triton X-100;
(c) the lysis buffer comprises a detergent which comprises Triton X-100 at a concentration of between 0.01% and 25%, between 0.01% and 10%, between 0.05% and 5%, between 0.1% and 2%, between 0.5% and 2%, between 0.75% and 1.25%, or about 1%;
(d) the lysis buffer comprises a detergent which comprises or consists of sodium deoxycholate;
(e) the lysis buffer comprises a detergent which comprises or consists of sodium deoxycholate at a concentration between 0.1% and 20%, between 0.1 and 10%, between 0.1 and 5%, between 0.5% and 5%, between 0.5% and 2.5%, between 0.75% and 2.5%, between 0.75% and 1.25%, or about 1%;
(f) the lysis buffer comprises a detergent which comprises or consists of sodium dodecyl sulphate (SDS);
(g) the lysis buffer comprises a detergent which comprises or consists of sodium dodecyl sulphate (SDS) at a concentration of between 0.001% and 10%, between 0.01% and 5%, between 0.05% and 5%, between 0.01% and 1%, between 0.05% and 1%, between 0.05% and 0.5%, between 0.075% and 0.25%, or about 0.1%;
(h) the lysis buffer comprises a lysis buffer which comprises a detergent which consists of Triton X-100, sodium deoxycholate, and sodium dodecyl sulphate (SDS);
(i) the lysis buffer comprises a detergent which consists of between 0.5% and 2% of Triton X-100, between 0.5% and 2% of sodium deoxycholate, and between 0.05% and 0.5% of sodium dodecyl sulphate (SDS);
(j) the lysis buffer comprises a buffer component;
(k) the lysis buffer comprises a buffer component which has a pH of between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6; and/or maintains the pH of the lysis buffer at between pH 4 and pH 9, between pH 5 and pH 8.5, between pH 6 and pH 8, between pH 6.5 and pH 8, between pH 7 and pH 8, between pH 7.3 and pH 7.9, between pH 7.4 and pH 7.8, between pH 7.5 and pH 7.7, or about pH 7.6;
(l) the lysis buffer comprises a buffer component which comprises or consists of Tris;
(m) the lysis buffer comprises a buffer component which comprises or consists of Tris at a concentration greater than 1 mM, between 1 mM and 350 mM, between 5 and 200 mM, between 5 and 100 mM, between 5 and 50 mM, between 5 mM and 40 mM, between 5 mM and 35 mM, between 10 mM and 35 mM, between 15 mM and 35 mM, between 15 mM and 30 mM, between 20 mM and 30 mM, or about 25 mM;
(n) the lysis buffer comprises a buffer component which consists of Tris at a concentration of between 15 mM and 35 mM;
(o) the lysis buffer comprises a salt;
(p) the lysis buffer comprises sodium chloride;
(q) the lysis buffer comprises sodium chloride at a concentration between 10 mM and 350 mM, between 20 mM and 300 mM, between 50 mM and 250 mM, between 100 mM and 250, between 100 mM and 200 mM, between 125 mM and 175 mM, or about 150 mM;
(r) the lysis buffer comprises sodium chloride at a concentration of between 100 mM and 200 mM;
(s) the lysis buffer comprises:
(i) between 1 mM and 100 mM Tris;
(ii) between 50 mM and 300 mM sodium chloride;
(iii) between 0.1 and 5% sodium deoxycholate;
(iv) between 0.01 and 1% sodium dodecyl sulphate; and/or
(v) between 0.1 and 5% Triton-X100; and/or
(t) the lysis buffer comprises:
(i) between 10 mM and 40 mM Tris, optionally about 25 mM Tris;
(ii) between 100 mM and 200 mM sodium chloride, optionally about 150 mM sodium chloride;
(iii) between 0.5 and 2% sodium deoxycholate, optionally about 1% sodium deoxycholate;
(iv) between 0.05 and 0.5% sodium dodecyl sulphate, optionally about 0.1% sodium dodecyl sulphate; and/or
(v) between 0.5 and 2% Triton-X100, optionally about 1% Triton-X100.

12. The method of any one of claims 2-11, wherein the cells which release the at least one biomarker are epithelial cells, optionally epithelial cells which have been shed from the lining of the prostate gland of the subject.

13. The method of any one of the preceding claims, wherein the step of detecting the at least one biomarker or determining the concentration of the at least one biomarker comprises:
exposing the semen sample to a first monoclonal antibody and/or a second monoclonal antibody; and
detecting the at least one biomarker bound to the first monoclonal antibody and/or the second monoclonal antibody or determining the concentration of the at least one biomarker bound to the first monoclonal antibody and/or the second monoclonal antibody, optionally wherein the first monoclonal antibody and the second monoclonal antibody bind to MCM5.

14. The method of claim 13, wherein the first monoclonal antibody is an antibody which:
(i) binds to a polypeptide having an amino acid sequence of SEQ ID NO: 1;
(ii) comprises at least one Complementary Determining Region (CDR) selected from the group consisting of:
(a) 12A7 CDRH1 which has a sequence of SEQ ID NO: 9 or a sequence that differs from SEQ ID NO:9 by a single amino acid substitution;
(b) 12A7 CDRH2 which has a sequence of SEQ ID NO: 11 or a sequence that differs from SEQ ID NO:11 by a single amino acid substitution;
(c) 12A7 CDRH3 which has a sequence of SEQ ID NO: 13 or a sequence that differs from SEQ ID NO:13 by a single amino acid substitution;
(d) 12A7 CDRL1 which has a sequence of SEQ ID NO: 3 or a sequence that differs from SEQ ID NO:3 by a single amino acid substitution;
(e) 12A7 CDRL2 which has a sequence of SEQ ID NO: 5 or a sequence that differs from SEQ ID NO:5 by a single amino acid substitution; and
(f) 12A7 CDRL3 which has a sequence of SEQ ID NO: 7 or a sequence that differs from SEQ ID NO:7 by a single amino acid substitution;
(iii) comprises a heavy chain variable region having a sequence at least 85%, 90%, 95%, 98%, 99%, or 100% identical to SEQ ID NO: 29;
(iv) comprises a light chain variable region sequence having a sequence at least 85%, 90%, 95%, 98%, 99%, or 100% identical to SEQ ID NO: 27; or
(v) competes with the antibody of (i), (ii), (iii), or (iv); and/or
the second monoclonal antibody is an antibody which:
(i) binds to a polypeptide having an amino acid sequence of SEQ ID NO: 2;
(ii) comprises at least one Complementary Determining Region (CDR) selected from the group consisting of:
(a) 4B4 CDRH1 which has a sequence of SEQ ID NO: 21 or a sequence that differs from SEQ ID NO:21 by a single amino acid substitution;
(b) 4B4 CDRH2 which has a sequence of SEQ ID NO: 23 or a sequence that differs from SEQ ID NO:23 by a single amino acid substitution;
(c) 4B4 CDRH3 which has a sequence of SEQ ID NO: 25 or a sequence that differs from SEQ ID NO:25 by a single amino acid substitution;
(d) 4B4 CDRL1 which has a sequence of SEQ ID NO: 15 or a sequence that differs from SEQ ID NO:15 by a single amino acid substitution;
(e) 4B4 CDRL2 which has a sequence of SEQ ID NO: 17 or a sequence that differs from SEQ ID NO:17 by a single amino acid substitution; and
(f) 4B4 CDRL3 which has a sequence of SEQ ID NO: 19 or a sequence that differs from SEQ ID NO:19 by a single amino acid substitution;
(iii) comprises a heavy chain variable region having a sequence at least 85%, 90%, 95%, 98%, 99%, or 100% identical to SEQ ID NO: 33;
(iv) comprises a light chain variable region sequence having a sequence at least 85%, 90%, 95%, 98%, 99%, or 100% identical to SEQ ID NO: 31; or
(v) competes with the antibody of (i), (ii), (iii), or (iv).

15. The method of claim 13 or 14, wherein:
(a) the first monoclonal antibody and/or second monoclonal antibody has an affinity for MCM5 in the range of 0.001-1 nM;
(b) the first monoclonal antibody and/or the second monoclonal antibody is a Fab′2, a F′(ab)₂, an Fv, a single chain antibody or a diabody;
(c) the first monoclonal antibody comprises 12A7 CDRH1, 12A7 CDRH2, and 12A7 CDRH3;
(d) the first monoclonal antibody comprises 12A7 CDRL1, 12A7 CDRL2, and 12A7 CDRL3;
(e) the first monoclonal antibody comprises a 12A7 CDRH1 which has a sequence of SEQ ID NO: 9, a 12A7 CDRH2 which has a sequence of SEQ ID NO: 11, and a 12A7 CDRH3 which has a sequence of SEQ ID NO: 13;
(f) the first monoclonal antibody comprises a 12A7 CDRL1 which has a sequence of SEQ ID NO: 3, a 12A7 CDRL2 which has a sequence of SEQ ID NO: 5, and a 12A7 CDRL3 which has a sequence of SEQ ID NO: 7;
(g) the second monoclonal antibody comprises 4B4 CDRH1, 4B4 CDRH2, and 4B4 CDRH3;
(h) the second monoclonal antibody comprises 4B4 CDRL1, 4B4 CDRL2 and 4B4 CDRL3;
(i) the second monoclonal antibody comprises a 4B4 CDRH1 which has a sequence of SEQ ID NO: 21, a 4B4 CDRH2 which has a sequence of SEQ ID NO: 23, and a 4B4 CDRH3 which has a sequence of SEQ ID NO: 25;
(j) the second monoclonal antibody has a 4B4 CDRL1 which has a sequence of SEQ ID NO: 15, a 4B4 CDRL2 which has a sequence of SEQ ID NO: 17, and a 4B4 CDRL3 which has a sequence of SEQ ID NO: 19;
(k) the first monoclonal antibody comprises a heavy chain variable region having a sequence at least 95% identical to SEQ ID NO: 29;
(l) the first monoclonal antibody comprises a heavy chain variable region having a sequence at least 98% identical to SEQ ID NO: 29;
(m) the first monoclonal antibody comprises a light chain variable region having a sequence at least 95% identical to SEQ ID NO: 27;
(n) the first monoclonal antibody comprises a light chain variable region having a sequence at least 98% identical to SEQ ID NO: 27;
(o) the second monoclonal antibody comprises a heavy chain variable region having a sequence at least 95% identical to SEQ ID NO: 33;
(p) the second monoclonal antibody comprises a heavy chain variable region having a sequence at least 98% identical to SEQ ID NO: 33; the second monoclonal antibody comprises a light chain variable region having a sequence at least 95% identical to SEQ ID NO: 31; and/or
(r) the second monoclonal antibody comprises a light chain variable region having a sequence at least 98% identical to SEQ ID NO: 31.

16. Use of a lysis buffer as defined in any one of the preceding claims in a method of detecting the presence of absence of prostate cancer in a subject.

17. Use of a first monoclonal antibody as defined in any one of the preceding claims and/or a second monoclonal antibody as defined in any one of the preceding claims in a method of detecting the presence or absence of prostate cancer in a subject.

18. Use of a kit in a method of detecting the presence or absence of prostate cancer in a subject, wherein the kit comprises:
(a) a lysis buffer as defined in any one of the preceding claims;
(b) a first monoclonal antibody as defined in any one of the preceding claims; and/or
(c) a second monoclonal antibody as defined in any one of the preceding claims.

19. A kit comprising:
(a) a lysis buffer as defined in any one of the preceding claims;
(b) a first monoclonal antibody as defined in any one of the preceding claims; and/or
(c) a second monoclonal antibody as defined in any one of the preceding claims; and
(d) instructions for use of the lysis buffer and/or the first monoclonal antibody and/or the second monoclonal antibody in a method of detecting the presence or absence of prostate cancer in a subject.

20. The method, use or kit of any one of the preceding claims, wherein the prostate cancer is grade 4/5.

21. The method, use or kit of any one of the preceding claims 1-20, wherein the prostate cancer is grade 1-3.


SEQUENCE LISTING (36 sequences)

SEQ ID NO: 1 - protein, 7 aa, Artificial sequence
  Epitope to which antibody 12A7 binds
WDETKGE

SEQ ID NO: 2 - protein, 7 aa, Artificial sequence
  Epitope to which antibody 4B4 binds
DDRVAIH

SEQ ID NO: 3 - protein, 11 aa, Artificial sequence
  12A7 light chain CDR 1 polypeptide sequence
QNLVQSNGNTY

SEQ ID NO: 4 - DNA, 36 bp, Artificial sequence
  12A7 light chain CDR 1 nucleotide sequence
cagaaccttgtacaaagtaatggaaacacctattta

SEQ ID NO: 5 - protein, 4 aa, Artificial sequence
  12A7 light chain CDR 2 polypeptide sequence
  misc_feature (4)..(4): Xaa can be any naturally occurring amino acid
KVSX

SEQ ID NO: 6 - DNA, 10 bp, Artificial sequence
  12A7 light chain CDR 2 nucleotide sequence
aagtttccaa

SEQ ID NO: 7 - protein, 9 aa, Artificial sequence
  12A7 light chain CDR 3 polypeptide sequence
SQSTRVPYT

SEQ ID NO: 8 - DNA, 27 bp, Artificial sequence
  12A7 light chain CDR 3 nucleotide sequence
tctcaaagtacacgtgttccgtacaca

SEQ ID NO: 9 - protein, 10 aa, Artificial sequence
  12A7 heavy chain CDR 1 polypeptide sequence
GFSLSTSGMG

SEQ ID NO: 10 - DNA, 30 bp, Artificial sequence
  12A7 heavy chain CDR 1 nucleotide sequence
gggttttcactgagcacttctggtatgggt

SEQ ID NO: 11 - protein, 7 aa, Artificial sequence
  12A7 heavy chain CDR 2 polypeptide sequence
IFWDDDK

SEQ ID NO: 12 - DNA, 21 bp, Artificial sequence
  12A7 heavy chain CDR 2 nucleotide sequence
attttctgggatgatgacaag

SEQ ID NO: 13 - protein, 13 aa, Artificial sequence
  12A7 heavy chain CDR 3 polypeptide sequence
ARRSDYNYYSMDY

SEQ ID NO: 14 - DNA, 39 bp, Artificial sequence
  12A7 heavy chain CDR 3 nucleotide sequence
gcgcggcgaagtgactacaattactactctatggactac

SEQ ID NO: 15 - protein, 6 aa, Artificial sequence
  4B4 light chain CDR 1 polypeptide sequence
QDIGSS

SEQ ID NO: 16 - DNA, 18 bp, Artificial sequence
  4B4 light chain CDR 1 nucleotide sequence
caggacattggtagtagc

SEQ ID NO: 17 - protein, 4 aa, Artificial sequence
  4B4 light chain CDR 2 polypeptide sequence
  misc_feature (4)..(4): Xaa can be any naturally occurring amino acid
ATSX

SEQ ID NO: 18 - DNA, 10 bp, Artificial sequence
  4B4 light chain CDR 2 nucleotide sequence
  misc_feature (10)..(10): n is a, c, g, or t
gccacatccn

SEQ ID NO: 19 - protein, 9 aa, Artificial sequence
  4B4 light chain CDR 3 polypeptide sequence
LQYASSPPT

SEQ ID NO: 20 - DNA, 27 bp, Artificial sequence
  4B4 light chain CDR 3 nucleotide sequence
ctacaatatgctagttctcctccgacg

SEQ ID NO: 21 - protein, 8 aa, Artificial sequence
  4B4 heavy chain CDR 1 polypeptide sequence
GFTFSNYA

SEQ ID NO: 22 - DNA, 24 bp, Artificial sequence
  4B4 heavy chain CDR 1 nucleotide sequence
ggattcactttcagtaactatgcc

SEQ ID NO: 23 - protein, 8 aa, Artificial sequence
  4B4 heavy chain CDR 2 polypeptide sequence
ISRGGSYT

SEQ ID NO: 24 - DNA, 24 bp, Artificial sequence
  4B4 heavy chain CDR 2 nucleotide sequence
attagtcgtggtggtagttacacc

SEQ ID NO: 25 - protein, 15 aa, Artificial sequence
  4B4 heavy chain CDR 3 polypeptide sequence
ARHGYNYDDGAWFAN

SEQ ID NO: 26 - DNA, 45 bp, Artificial sequence
  4B4 heavy chain CDR 3 nucleotide sequence
gcaagacatggatataattacgacgacggggcctggtttgctaac

SEQ ID NO: 27 - protein, 113 aa, Artificial sequence
  12A7 full variable light chain sequence (polypeptide)
DIMLTQSPLSLSVTLGDQASISCRSSQNLVQSNGNTYLTWYLQKPGQSPKVLINKVSNRFYGVPDRFSGSGSGTDFTLRISRVEAEDLGIYFCSQSTRVPYTFGGGTKLEIRR

SEQ ID NO: 28 - DNA, 338 bp, Artificial sequence
  12A7 full variable light chain sequence (nucleotide)
gatatcatgctgacccaatctccactctccctgtctgtcactcttggagatcaggcctccatctcttgcagatctagtcagaaccttgtacaaagtaatggaaacacctatttaacttggtacctgcagaagccaggccagtctccaaaggtcctgatcaacaaagtttccaaccgattttatggggtcccagacaggttcagtggcagtggatcagggacagatttcacactcaggatcagcagagtggaggctgaggatctgggaatttatttctgctctcaaagtacacgtgttccgtacacattcggaggggggaccaagctggaaataagacg

SEQ ID NO: 29 - protein, 121 aa, Artificial sequence
  12A7 full variable heavy chain sequence (polypeptide)
QQDLQQSGPGILQPTQTLSLTCSFSGFSLSTSGMGVSWIRQSSNMGLEWLAHIFWDDDKRYNPSLRSRLTLSKDTSSSQVFLMITSVSTADSATYYCARRSDYNYYSMDYWGQGTAVTVSS

SEQ ID NO: 30 - DNA, 363 bp, Artificial sequence
  12A7 full variable heavy chain sequence (nucleotide)
cagcaagatctgcagcagtctggccctgggatattgcagcccacccagaccctcagtctgacttgttctttctctgggttttcactgagcacttctggtatgggtgtgagttggattcgtcaatcttcaaatatgggtctggagtggctggcacacattttctgggatgatgacaagcgctataatccctccctgaggagccgactcacgctctccaaggatacctccagtagccaggtatttctcatgatcaccagtgtgagtactgcagattctgccacatactactgtgcgcggcgaagtgactacaattactactctatggactactggggtcaaggaaccgcagtcaccgtctcctca

SEQ ID NO: 31 - protein, 107 aa, Artificial sequence
  4B4 full variable light chain sequence (polypeptide)
DIMLTQSPSSLSASLGERVSLTCRASQDIGSSLNWLQQEPDGTIKRLIYATSSLDSGVPKRFSGSRSGSDYSLTISSLESEDFVDYYCLQYASSPPTFGGGTKLEIK

SEQ ID NO: 32 - DNA, 322 bp, Artificial sequence
  4B4 full variable light chain sequence (nucleotide)
gatatcatgctgacccaatctccatcctccttatctgcctctctgggagaaagagtcagtctcacttgtcgggcaagtcaggacattggtagtagcttaaactggcttcaacaggaaccagatggaactattaaacgcctaatctacgccacatccagtttagattctggtgtccccaaaaggttcagtggcagtaggtctgggtcagattattctctcaccatcagcagccttgagtctgaagattttgtagactattactgtctacaatatgctagttctcctccgacgttcggtggaggcaccaagctggaaatcaaac

SEQ ID NO: 33 - protein, 121 aa, Artificial sequence
  4B4 full variable heavy chain sequence (polypeptide)
VKLQESGGGLVKPGGSLKLSCAASGFTFSNYAMSWVRQNPEKRLEWVATISRGGSYTYYPDSVKGRFTISRDNAKNTLYLQMNSLRSEDTAMYFCARHGYNYDDGAWFANWGQGTLVTVSA

SEQ ID NO: 34 - DNA, 366 bp, Artificial sequence
  4B4 full variable heavy chain sequence (nucleotide)
taggtgaaactgcaggagtctgggggaggcttagtgaagcctggagggtccctgaaactctcctgtgcagcctctggattcactttcagtaactatgccatgtcttgggttcgccagaatccggagaagaggctggagtgggtcgcaaccattagtcgtggtggtagttacacctactatccagacagtgtgaagggtcgattcaccatctccagagacaatgccaagaacaccctgtatctgcaaatgaacagtctgaggtctgaggacacggccatgtatttctgtgcaagacatggatataattacgacgacggggcctggtttgctaactggggccaagggactctggtcactgtctctgca

SEQ ID NO: 35 - protein, 734 aa, Homo sapiens
  Mcm5 (polypeptide)
MSGFDDPGIFYSDSFGGDAQADEGQARKSQLQRRFKEFLRQYRVGTDRTGFTFKYRDELKRHYNLGEYWIEVEMEDLASFDEDLADYLYKQPAEHLQLLEEAAKEVADEVTRPRPSGEEVLQDIQVMLKSDASPSSIRSLKSDMMSHLVKIPGIIIAASAVRAKATRISIQCRSCRNTLTNIAMRPGLEGYALPRKCNTDQAGRPKCPLDPYFIMPDKCKCVDFQTLKLQELPDAVPHGEMPRHMQLYCDRYLCDKVVPGNRVTIMGIYSIKKFGLTTSRGRDRVGVGIRSSYIRVLGIQVDTDGSGRSFAGAVSPQEEEEFRRLAALPNVYEVISKSIAPSIFGGTDMKKAIACLLFGGSRKRLPDGLTRRGDINLLMLGDPGTAKSQLLKFVEKCSPIGVYTSGKGSSAAGLTASVMRDPSSRNFIMEGGAMVLADGGVVCIDEFDKMREDDRVAIHEAMEQQTISIAKAGITTTLNSRCSVLAAANSVFGRWDETKGEDNIDFMPTILSRFDMIFIVKDEHNEERDVMLAKHVITLHVSALTQTQAVEGEIDLAKLKKFIAYCRVKCGPRLSAEAAEKLKNRYIIMRSGARQHERDSDRRSSIPITVRQLEAIVRIAEALSKMKLQPFATEADVEEALRLFQVSTLDAALSGTLSGVEGFTSQEDQEMLSRIEKQLKRRFAIGSQVSEHSIIKDFTKQKYPEHAIHKVLQLMLRRGEIQHRMQRKVLYRLK

SEQ ID NO: 36 - DNA, 2534 bp, Homo sapiens
  Mcm5 (mRNA)
ggaaaaccagaggcgcagtcatgtcgggattcgacgatcctggcattttctacagcgacagcttcgggggcgacgcccaggccgacgaggggcaggcccgcaaatcgcagctgcagaggcgcttcaaggagttcctgcggcggtaccgagtgggcaccgaccgcacgggcttcaccttcaaatacagggatgaactcaagcggcattacaacctgggggagtactggattgaggtggagatggaggatctggccagctttgatgaggacctggccgactacttgtacaagcagccagccgagcacctgcagctgctggaggaagctgccaaggaggtagctgatgaggtgacccggccccggccttctggggaggaggtgctccaggacatccaggtcatgctcaagtcggacgccagcccttccagcattcgtagcctgaagtcggacatgatgtcacacctggtgaagatccctggcatcatcatcgcggcctctgcggtccgtgccaaggccacccgcatctctatccagtgccgcagctgccgcaacaccctcaccaacattgccatgcgccctggcctcgagggctatgccctgcccaggaagtgcaacacagatcaggctgggcgccccaaatgcccattggacccgtacttcatcatgcccgacaaatgcaaatgcgtggacttccagaccctgaagctgcaggagctgcctgatgcagtcccccacggggagatgcccagacacatgcagctctactgcgacaggtacctgtgtgacaaggtcgtccctgggaacagggttaccatcatgggcatctactccatcaagaagtttggcctgactaccagcaggggccgtgacagggtgggcgtgggcatccgaagctcctacatccgtgtcctgggcatccaggtggacacagatggctctggccgcagctttgctggggccgtgagcccccaggaggaggaggagttccgtcgcctggctgccctcccaaatgtctatgaggtcatctccaagagcatcgccccctccatctttgggggcacagacatgaagaaggccattgcctgcctgctctttgggggctcccgaaagaggctccctgatggacttactcgccgaggagacatcaacctgctgatgctaggggaccctgggacagccaagtcccagcttctgaagtttgtggagaagtgttctcccattggggtatacacgtctgggaaaggcagcagcgcagctggactgacagcctcggtgatgagggacccttcgtcccggaatttcatcatggagggcggagccatggtcctggccgatggtggggtcgtctgtattgacgagtttgacaagatgcgagaagatgaccgtgtggcaatccacgaagccatggagcagcagaccatctctatcgccaaggctgggatcaccaccaccctgaactcccgctgctccgtcctggctgctgccaactcagtgttcggccgctgggatgagacgaagggggaggacaacattgacttcatgcccaccatcttgtcgcgcttcgacatgatcttcatcgtcaaggatgagcacaatgaggagagggatgtgatgctggccaagcatgtcatcactctgcacgtgagcgcactgacacagacacaggctgtggagggcgagattgacctggccaagctgaagaagtttattgcctactgccgagtgaagtgtggcccccggctgtcagcagaggctgcagagaaactgaagaaccgctacatcatcatgcggagcggggcccgtcagcacgagagggacagtgaccgccgctccagcatccccatcactgtgcggcagctggaggccattgtgcgcatcgcggaagccctcagcaagatgaagctgcagcccttcgccacagaggcagatgtggaggaggccctgcggctcttccaagtgtccacgttggatgctgccttgtccggtaccctgtcaggggtggagggcttcaccagccaggaggaccaggagatgctgagccgcatcgagaagcagctcaagcgccgctttgccattggctcccaggtgtctgagcacagcatcatcaaggacttcaccaagcagaaatacccggagcacgccatccacaaggtgctgcagctcatgctgcggcgcggcgagatccagcatcgcatgcagcgcaaggttctctaccgcctcaagtgagtcgcgccgcctcactggactcatggactcgcccacgcctcgcccctcctgccgctgcctgccattgacaatgttgctgggacctctgcctccccactgcagccctcgaacttcccaggcaccctcctttctgccccagaggaaggagctgtagtgtcctgctgcctctgggcgcccgcctctagcgcggttctgggaagtgtgcttttggcatccgttaataataaagccacggtgtgttcaggtaaaaaaaaaaaaaaaaaaaaaaaaaaaaaaaaaaaaaaaaaaaaaaaaaaaaaaaaaaaaaaa